(19) United States
(12) Patent Application Publication
(10) Pub. No.: US 2014/0058000 A1
(43) Pub. Date: Feb. 27, 2014

(54) DIAGNOSTIC MARKERS FOR DETERMINING THE PREDISPOSITION TO THE DEVELOPMENT OF CERVICAL CANCER AND OLIGONUCLEOTIDES USED FOR THE DETERMINATION

(71) Applicant: Eberhard-Karls-Universitaet Tuebingen Universitaet Universitaetsklinikum, Tuebingen (DE)

(72) Inventors: Thomas Iftner, Hirrlingen (DE); Frank Stubenrauch, Tuebingen (DE); Anna Manawapat, Tuebingen (DE); Susanne Krueger Kjaer, Virum (DK)

(73) Assignee: Eberhard-Karls-Universitaet Tuebingen Universitaet Universitaetsklinikum, Tuebingen (DE)

(21) Appl. No.: 14/071,470

(22) Filed: Nov. 4, 2013

Related U.S. Application Data
(63) Continuation of application No. 13/294,905, filed on Nov. 11, 2011.
Application No. 13/294,905 is a continuation of application No. PCT/EP2010/056595, filed on May 12, 2010.

(30) Foreign Application Priority Data
May 12, 2009 (DE) 102009021734.7

(51) Int. Cl. C12Q 1/68; G01N 33/574

(52) U.S. Cl. CPC C12Q 1/6886; G01N 33/57411
USPC 514/789; 435/5; 506/9; 536/24.31

(57) ABSTRACT

The present invention relates to a method for predisposition prediction of a subject to the development of cervical cancer and/or cancer precursors in an infection with the human papilloma virus (HPV) and/or for the detection of a persistent HPV infection, the method comprising the steps of obtaining a sample from the subject; and detecting at least one of the diagnostic markers or fragments thereof listed in Table 1 in the sample obtained from the subject.

### CROSS REFERENCES TO RELATED APPLICATIONS

This application is a continuation of U.S. application Ser. No. 13/294,905, filed on Nov. 11, 2011, which is a continuation of international patent application number PCT/EP2010/056595, filed on May 12, 2010, designating the U.S., which international patent application has been published in German language as WO 2010/130809 A1 and claims priority from German patent application number DE 10 2009 021 734.7 filed on May 12, 2009. The entire contents of these priority applications are incorporated herein by reference.

### BACKGROUND OF THE INVENTION

The present invention relates to the use of diagnostic markers in a method for the prediction of the predisposition of a person to the development of cervical cancer and/or cancer precursors in the case of an infection with the human papilloma virus, and for the detection of a persistent infection with the human papilloma virus.

The invention furthermore relates to a method for prediction of the predisposition of a person to the development of cervical cancer and/or cancer precursors in an infection with the human papilloma virus (below also: HPV), in which diagnostic markers are detected, as well as the provision of oligonucleotides suitable for this.

Papilloma viruses are widespread, small double-stranded DNA viruses that can infect cells in the basal layer of the epidermis or of the cervical epithelium, inter alia of humans. Whilst most infections are temporally restricted and have an asymptomatic course, a persistent infection of the genital mucosa with certain papilloma viruses can lead to a formation of a cervical intraepithelial neoplasia (CIN), which in turn can develop into a cervical carcinoma.

Cervical cancer is the second most frequent cancer in women worldwide, and the worldwide prevalence of human papilloma viruses in cervical carcinomas is 99.7%. Infection with certain genital HPV types is a necessary risk factor for the development of a cervical carcinoma.

Although HPV infections are very frequent, an infection in most cases runs a transient course and is combated spontaneously. The persistence of an HPV infection is a necessary risk factor for the development and progression of cervical precancerous lesions, but only about 10% of the women with an HPV infection show a progression to such lesions; of these about 20 to 50% of the women develop, untreated, depending on the persistent HPV type, cancer precursors or cervical cancer within a period of up to 12 years.

Presently, no diagnostic tests are known in the prior art that discriminate between women persistently infected with HPV and women who remain healthy.

The early recognition of cancer up to now detects cancer precursors by cytological screening, in which a smear is taken from the mouth of the uterus and the endocervical canal, stained according to Papanicolaou and assessed microscopically (Papanicolaou smear or Pap test). In addition, there are tests for the detection of human papilloma viruses at the DNA and RNA level which, however, cannot differentiate between the frequent transient infections and the rather rarer persistent infections and therefore only have a low positive prediction value (PPV) of 15 to 25% for cancer precursors.

Starting from such an HPV test alone, many women would be or are sent for follow-up investigations, which results in increased overtreatment.

At the protein level, the genetic marker p16 is described, which is a surrogate marker for the HPV infection, which in studies, however, shows too low a specificity and likewise only a small PPV for cancer precursors.

### SUMMARY OF THE INVENTION

An object of the present invention is therefore to make available a test with the aid of which the predisposition of a person to the development of cervical cancer can be predicted in an infection with the human papilloma virus.

According to the invention, this and other objects are achieved by a method for predisposition prediction of a subject to the development of cervical cancer and/or cancer precursors in an infection with the human papilloma virus (HPV) and/or for the detection of a persistent infection with the human papilloma virus (HPV), the method comprising the steps of obtaining a sample from the subject and detecting at least one of the diagnostic markers or fragments thereof listed in Table 1 in the sample obtained from the subject. In particular, the method comprises the steps of obtaining a sample from the subject and determining the amount of, or relative amount of, one or more diagnostic markers/species in the sample; and relating the amount of, or relative amount of, the one or more diagnostic markers/species present in said sample, as compared to a control sample.

TABLE 1
Diagnostic markers

| Gene Symbol | Gene Title |
|---|---|
| ABCA1 | ATP-binding cassette, sub-family A (ABC1), |
| | member 1 |
| ACSL1 | acyl-CoA synthetase long-chain family member 1 |
| ACSL5 | acyl-CoA synthetase long-chain family member 5 |
| ADORA2A | adenosine A2a receptor |
| AGFG2 | ArfGAP with FG repeats 2 |
| AIG1 | androgen-induced 1 |
| AIP | aryl hydrocarbon receptor interacting protein |
| ALDOB | Aldolase B, fructose-bisphosphate, mRNA |
| | (cDNA clone MGC:32618 IMAGE:4593670) |
| ANKRD13D | ankyrin repeat domain 13 family, member D |
| APTX | Aprataxin |
| AQP3 | aquaporin 3 (Gill blood group) |
| ARHGAP15 | Rho GTPase activating protein 15 |
| ARHGAP26 | Rho GTPase activating protein 26 |
| ARMC8 | armadillo repeat containing 8 |
| ARPC5L | actin related protein 2/3 complex, subunit 5-like |
| ASAP1 | ArfGAP with SH3 domain, ankyrin repeat and PH |
| | domain 1 |
| ATF7 | activating transcription factor 7 |
| ATP2B1 | ATPase, Ca++ transporting, plasma membrane 1 |
| ATP2B1 | ATPase, Ca++ transporting, plasma membrane 1 |
| ATP6V1C1 | ATPase, H+ transporting, lysosomal 42 kDa, V1 |
| | subunit C1 |
| ATP9A | ATPase, class II, type 9A |
| BCL6 | B-cell CLL/lymphoma 6 |
| BEST1 | bestrophin 1 |
| BIC | BIC transcript |
| BID | BH3 interacting domain death agonist |
| BRE | brain and reproductive organ-expressed |
| | (TNFRSF1A modulator) |
| C12orf35 | chromosome 12 open reading frame 35 |
| C17orf91 | chromosome 17 open reading frame 91 |
| C19orf66 | chromosome 19 open reading frame 66 |
| C22orf37 | chromosome 22 open reading frame 37 |
| C9orf72 | chromosome 9 open reading frame 72 |
| CAMKK2 | calcium/calmodulin-dependent protein kinase kinase |
| | 2, beta |
| CAND1 | Cullin-associated and neddylation-dissociated 1 |
| CARD16 | caspase recruitment domain family, member 16 |
| CARD8 | caspase recruitment domain family, member 8 |
| CCL18 | chemokine (C-C motif) ligand 18 (pulmonary and |
| | activation-regulated) |
| CCRL2 /// LOC72 | chemokine (C-C motif) receptor-like 2 /// similar to |
| | chemokine (C-C motif) receptor-like |
| CD2AP | CD2-associated protein |
| CD44 | CD44 molecule (Indian blood group) |
| CD83 | CD83 molecule |
| CDC42EP3 | CDC42 effector protein (Rho GTPase binding) 3 |
| CHD1 | chromodomain helicase DNA binding protein 1 |
| CLCA2 | Chloride Channel regulator 2 |
| CPSF2 | cleavage and polyadenylation specific factor |
| | 2,100 kDa |
| CREM | cAMP responsive element modulator |
| CSGALNACT2 | chondroitin sulfate N-acetylgalactosaminyltransferase |
| | 2 |
| CTNNA1 | catenin (cadherin-associated protein), alpha 1, |
| | 102 kDa |
| CTSD | cathepsin D |
| CXorf21 | chromosome X open reading frame 21 |
| CXXC5 | CXXC finger 5 |
| CYLC1 | Cylicin, basic protein of sperm head cytoskeleton 1 |
| DSC3 | desmocollin 3 |
| DSE | dermatan sulfate epimerase |
| DSG3 | desmoglein 3 (Pemphigus vulgaris antigen) |
| DYNC1H1 | dynein, cytoplasmic 1, heavy chain 1 |
| EHF | Ets homologous factor, mRNA (cDNA clone |
| | MGC:47678 IMAGE:6055934) |
| ELF1 | E74-Iike factor 1 (ets domain transcription factor) |
| ELL2 | elongation factor, RNA Polymerase II, 2 |
| EMR1 | egf-iike module containing, mucin-like, hormone |
| | receptor-like 1 |
| ERMP1 | endoplasmic reticulum metallopeptidase 1 |
| FBX045 | F-box protein 45 |
| FLJ44342 | hypothetical LOC645460 |
| FLNA | filamin A, alpha (actin binding protein 280) |
| FNDC3B | fibronectin type III domain containing 3B |
| FTL | ferritin, light Polypeptide |
| GBP1 | guanylate binding protein 1, interferon-inducible, |
| | 67 kDa |
| GBP5 | guanylate binding protein 5 |
| GCNT7 | Glucosaminyl (N-acetyl) transferase family member 7 |
| | (GCNT7), mRNA |
| GEMIN4 | Gern (nuclear organelle) associated protein 4 |
| GK | glycerol kinase |
| GK3P | glycerol kinase 3 pseudogene |
| GLS2 | glutaminase 2 (liver, mitochondrial) |
| GNA13 | guanine nucleotide binding protein (G protein), |
| | alpha 13 |
| GNB1 | guanine nucleotide binding protein (G protein), |
| | beta Polypeptide 1 |
| GNG2 | guanine nucleotide binding protein (G protein), |
| | gamma 2 |
| GPR84 | G protein-coupled receptor 84 |
| GRAMD1A | GRAM domain containing 1A, mRNA (cDNA clone |
| | IMAGE:5921205) |
| GRB2 | growth factor receptor-bound protein 2 |
| HAS2 | hyaluronan synthase 2 |
| HBB | hemoglobin, beta |
| HIF3A | hypoxia inducible factor 3, alpha subunit |
| HK3 | hexokinase 3 (white cell) |
| HMCN2 | CDNA FLJ23816 fis, clone HSI02685 |
| HN1L | hematological and neurological expressed 1-like |
| HSPA6 | Heat shock 70 kDa protein 6 (HSP70B′) |
| IFNGR2 | interferon gamma receptor 2 (interferon gamma |
| | transducer 1) |
| IL18BP | interleukin 18 binding protein |
| 1L20RB | interleukin 20 receptor beta |
| 1L7R | interleukin 7 receptor |
| INHB A | inhibin, beta A |
| INSIG1 | insulin induced gene 1 |
| IQCA1 | IQ motif containing with AAA domain 1 |
| IQCE | IQ motif containing E |
| IRAK3 | interleukin-1 receptor-associated kinase 3 |
| ITGA5 | integrin, alpha 5 (fibronectin receptor, alpha |
| | Polypeptide) |
| ITGB3 | integrin, beta 3 (platelet glycoprotein lila, antigen |
| | CD61) |
| ITGB5 | integrin, beta 5 |
| IVNS1ABP | influenza virus NS1A binding protein |
| JMJD3 | jumonji domain containing 3, histone lysine |
| | demethylase |
| KIAA0999 | KIAA0999 protein |
| LOCI 00128821 | /// hypothetical protein LOCI00128821 |
| | /// zinc finger E-box binding homeobox 2 |
| LOCI 42937 | hypothetical protein BC008131 |
| LOC338758 | hypothetical protein LOC338758 |
| LOC728153 | similar to FAM133B protein |
| LPCAT1 | lysophosphatidylcholine acyltransferase 1 |
| LRP11 | low density lipoprotein receptor-related protein 11 |
| LYN | v-yes-1 Yamaguchi sarcoma viral related oncogene |
| | homolog |
| LYST | lysosomal trafficking regulator |
| MAL2 | mal, T-cell differentiation protein 2 |
| MALAT1 | metastasis associated lung adenocarcinoma transcript |
| | 1 (non-protein coding) |
| MAN2A1 | mannosidase, alpha, class 2A, member 1 |
| MAP3K8 | CDNA FLJ78064 complete cds, highly similar to |
| | Human mRNA for proto-oncogene protein |
| MAP4K4 | mitogen-activated protein kinase kinase kinase |
| | kinase 4 |
| MCOLN2 | mucolipin 2 |
| METAP2 | methionyl aminopeptidase 2 |
| MLL5 | myeloid/lymphoid or mixed-lineage leukemia 5 |
| | (trithorax homolog, Drosophila) |
| M0BKL1B | MOB1, Mps One Binder kinase activator-like 1B |
| | (yeast) |
| MRPS28 | mitochondrial ribosomal protein S28 |
| MRPS7 | mitochondrial ribosomal protein S7 |
| MTF1 | metal-regulatory transcription factor 1 |
| NAMPT | nicotinamide phosphoribosyltransferase |
| NBN | Nibrin |
| NFKB 1 | nuclear factor of kappa light Polypeptide gene |
| | enhancer in B-cells 1 |
| NFKBIE | nuclear factor of kappa light Polypeptide gene |
| | enhancer in B-cells inhibitor, epsilon |
| NFKBIZ | nuclear factor of kappa light Polypeptide gene |
| | enhancer in B-cells inhibitor, zeta |
| NKX3-1 | NK3 homeobox 1 |
| NLRP3 | NLR famiiy, pyrin domain containing 3 |
| NR4A2 | nuclear receptor subfamily 4, group A, member 2 |
| NR4A3 | nuclear receptor subfamily 4, group A, member 3 |
| NSMAF | neutral sphingomyelinase (N-SMase) activation |
| | associated factor |
| 0R1F1 | olfactory receptor, famiiy 1, subfamily F, member 1 |
| PAG1 | phosphoprotein associated with glycosphingolipid |
| | microdomains 1 |
| PCDH1 | protocadherin 1 |
| PCY0X1 | prenylcysteine oxidase 1 |
| PERP | PERP, TP53 apoptosis effector |
| PHACTR1 | Phosphatase and actin regulator 1 |
| PHF20L1 | PHD finger protein 20-like 1 |
| PIK3AP1 | phosphoinositide-3-kinase adaptor protein 1 |
| PIK3R5 | phosphoinositide-3-kinase, regulatory subunit 5 |
| PNPLA8 | patatin-like phospholipase domain containing 8 |
| POLH | Polymerase (DNA directed), eta |
| POU6F1 | POL) class 6 homeobox 1 |
| PPP1R10 | protein Phosphatase 1, regulatory (inhibitor) |
| | subunit 10 |
| PPP2R3C | protein Phosphatase 2 (formerly 2A), regulatory |
| | subunit B″, gamma |
| PROM2 | prominin 2 |
| PRR14 | Proline rich 14 |
| PTGER4 | Prostaglandin E receptor 4 (subtype EP4) |
| PTK2 | PTK2 protein tyrosine kinase 2 |
| PTPRE | protein tyrosine Phosphatase, receptor type, E |
| PTX3 | pentraxin-related gene, rapidly induced by IL-1 beta |
| QKI | quaking homolog, KH domain RNA binding (mouse) |
| RAB8B | RAB8B, member RAS oncogene famiiy |
| RAP2C | RAP2C, member of RAS oncogene famiiy |
| RBX1 | ring-box 1 |
| REG4 | regenerating islet-derived famiiy, member 4 |
| RGS1 | regulator of G-protein signaling 1 |
| RHOQ | ras homolog gene famiiy, member Q |
| RIPK2 | receptor-interacting serine-threonine kinase 2 |
| RNF213 | ring finger protein 213 |
| RPP21 /// TRIM39 | ribonuclease P/MRP 21 kDa subunit |
| | /// TRIM39-Iike protein |
| SAMSN1 | SAM domain, SH3 domain and nuclear localization |
| | Signals 1 |
| SC02 | SCO cytochrome oxidase deficient homolog 2 (yeast) |
| SDC1 | syndecan 1 |
| SEC14L1 | SEC14-Iike 1 (S. cerevisiae) |
| SERPINB5 | serpin peptidase inhibitor, clade B (ovalbumin), |
| | member 5 |
| SERPINB9 | serpin peptidase inhibitor, clade B (ovalbumin), |
| | member 9 |
| SF3B1 | splicing factor 3b, subunit 1, 155 kDa |
| SF3B3 | splicing factor 3b, subunit 3, 130 kDa |
| SFRS2IP | splicing factor, arginine/serine-rich 2, interacting |
| | protein |
| SH3BP5 | SH3-domain binding protein 5 (BTK-associated) |
| SLC16A10 | solute carrier family 16, member 10 (aromatic amino |
| | acid transporter) |
| SLC25A33 | solute carrier family 25, member 33 |
| SLC25A37 | solute carrier family 25, member 37 |
| | CDNA FLJ90227 fis, clone NT2RM1000899, highly |
| | similar to Mitochondrial solute carrier protein |
| SNX10 | sorting nexin 10 |
| S0D2 | Superoxide dismutase 2, mitochondrial |
| S0X15 | SRY (sex determining region Y)-box 15 |
| SPAG9 | sperm associated antigen 9 |
| SRA1 | Steroid receptor RNA activator 1 |
| SRGN | Serglycin |
| STX4 | syntaxin 4 |
| SUMF1 | sulfatase modifying factor 1 |
| TACSTD2 | tumor-associated calcium Signal transducer 2 |
| TANK | TRAF family member-associated NFKB activator |
| TFEC | transcription factor EC |
| TGFB1 | transforming growth factor, beta 1 |
| TLR10 | toll-like receptor 10 |
| TMED3 | transmembrane emp24 protein transport domain |
| | containing 3 |
| TMEM45A | transmembrane protein 45A |
| TMEM87A | transmembrane protein 87A |
| TNFAIP3 | tumor necrosis factor, alpha-induced protein 3 |
| TNFAIP6 | tumor necrosis factor, alpha-induced protein 6 |
| TNFSF10 | tumor necrosis factor (ligand) superfamily, member 10 |
| TNFSF8 | tumor necrosis factor (ligand) superfamily, member 8 |
| TNK2 | tyrosine kinase, non-receptor, 2 |
| TPD52L1 | tumor protein D52-Iike 1 |
| TRAF1 | TNF receptor-associated factor 1 |
| TRIM38 | tripartite motif-containing 38 |
| UMPS | uridine monophosphate synthetase |
| UNC13A | unc-13 homolog A (C. elegans) |
| USP36 | ubiquitin specific peptidase 36 |
| WBSCR22 | Williams Beuren Syndrome chromosome region 22 |
| WDR43 | WD repeat domain 43 |
| WTAP | Wilms tumor 1 associated protein |
| WTAP | Wilms tumor 1 associated protein (WTAP), transcript |
| | variant 2, mRNA |
| ZBTB 11 | zinc finger and BTB domain containing 11 |
| ZEB2 | zinc finger E-box binding homeobox 2 |
| ZMYM6 | zinc finger, MYM-type 6 |
| ZNF267 | zinc finger protein 267 |
| ZNF438 | zinc finger protein 438 |
| ZSWIM7 | zinc finger, SWIM-type containing 7 |
| ZXDB | zinc finger, X-Iinked, duplicated B |

Furthermore, an object is achieved by providing oligonucleotides that are employed during the method according to the invention, and with which at least one of the diagnostic markers shown in Table 1 can be detected, and by a kit for carrying out the method according to the invention, which contains at least one oligonucleotide according to the invention.

The inventors were able to show in elaborate experiments that it is possible by means of the markers investigated, for example, to detect these markers in a sample taken from a person to be investigated, and in their presence, either alone or in combination with one or more of the other markers listed, to determine a predisposition of the person for the development of cervical cancer. This determination can be carried out, for example, by means of the comparison of the markers according to the invention with the corresponding markers from corresponding HPV-negative samples and/or HPV-positive, but not progressive, samples.

An excellent tool for improved early recognition of cervical cancer is therefore provided by the present invention, since with the aid of the markers identified in the present invention, the development of high-grade cancer precursors can be predicted with high significance up to 12 years beforehand on the basis of a persistent HPV infection.

Furthermore, the markers prepared, in addition to the possibility of discrimination between women with a progressive course and women who, despite an HPV infection remain healthy, also provide the possibility of differentiating between women who exhibit no HPV infection and women who have a persistent HPV infection.

Thus the risk of women developing cervical cancer, starting from a persistent HPV infection, can also be predicted.

The terms “advancing” and “progressive” are used synonymously here, and are intended to mean—as also is the term “progressors”—the development of dysplasias or cancer precursors and cancers/tumors.

The names rendered in Table 1 are the official gene names—also in Germany—, or their official abbreviation, their translation into German is neither appropriate nor recommended, in order to avoid confusion. Further information on the genes mentioned, their sequences and names are to be found by means of their given abbreviations, for example, in the database of the National Center for Biotechnology Information (http://www.ncbi.nlm.nih.gov/science/entrez) under the search tool “genes”, where the information is located in a publicly accessible and permanently stored form.

It is in particular preferred here if the markers are selected from the group comprising the markers with the official gene names SERPINB5, CNAD1, CPSF2, CCRL2, TNFAIP6, CD44, TMEM45A, WDR43, NBN, ERMP1, LRP11, CDKN2A, HPV16E4, HPV16E7, SERP1, ASAH1, HIGD1A, PGH1, which are also listed in Table 2 below with their respective sequences, and the primers in each case specific for the markers (see also Table 3) and the respective product size.

TABLE 2
selected diagnostic markers

| Marker | Sequence | Primer |
|---|---|---|
| SERPINB5 | cttcgttcgcagagcttttcagattgtggaatgttggataaggaattatagacctctagta | forward Primer |
| serpin peptidase | gctgaaatgcaagaccccaagaggaagttcagatcttaatataaattcactttcatttttg | gttgccggttcatggatt |
| inhibitor, clade | atagctgtcccatctggtcatgtggttggcactagactggtggcaggggcttctagctg | ac (SEQ ID NO: |
| B | actcgcacagggattctcacaatagccgatatcagaatttgtgttgaaggaacttgtctc | 2) |
| (also known as | ttcatctaatatgatagcgggaaaaggagaggaaactactgcctttagaaaatataagt | reverse Primer |
| PI5; maspin) | aaagtgattaaagtgctcacgttaccttgacacatagtttttcagtctatgggtttagttact | gcatgtcaaggaagag |
| | ttagatggcaagcatgtaacttatattaatagtaatttgtaaagttgggtggataagctatc | atgg (SEQ ID NO. |
| | cctgttgccggttcatggattacttctctataaaaaatatatatttaccaaaaaattttgtga | 3) |
| | cattccttctcccatctcttccttgacatgcattgtaaataggttcttcttgttctgag | product size |
| | (SEQ ID NO: 1) | 91 bp |
| CAND1 | Gaggcagttgttaaattgagtgaccaatttaagcaatcagatatttgaaaactgcaccc | forward Primer |
| cullin-associated | tttagttttgaaactgtgaattagaaacacttttcctgctgtattactacctgctttaacatcc | gcttgattttgaatgttca |
| and neddylation- | aaatatacagtgattttaaatgataacatactgtggttattagattaacagcttgattttgaa | gatg (SEQ ID |
| dissociated 1 | tgttcagatgataatgcagaagacatcacttctagtaaggattttgactagtgcattgatg | NO: 5) |
| (also known as | ttgaagttggtgccatttcaaaatgtggcaggtgataatcttttaccataatttgcataaaa | reverse Primer |
| TIP120; | ctgtaatagaagtttattttgagatgttagtatattatgtactatgcatttct9gtggtata | gattatcacctgccacat |
| T1P120A; | (SEQ ID NO: 4) | tttg (SEQ ID NO: |
| FLJ10114; | | 6) |
| FLJ10929; | | Product size |
| FLJC) | | 114 bp |
| CPSF2 | aagcatacgtgctatgactctctccagtttgaatatgcaattgttttcacaggcaggatgt | forward Primer |
| cleavage and | ctgttttctgcctgtatttcccagtgatttactctagggtaaggtagtacacatttggttcag | gccgtaagttaggtcct |
| polyadenylation | aaattaatttttatttctcctatatcttgttttatcaagattttgttgtggcatttcaatgtaaa | gtgtt (SEQ ID |
| specific factor 2, | ttataacaccannnnnntcatttgagtatacataattcaaaagaactacttgatgcagtata | NO: 8) |
| 10OkDa | gtcttaagggttctgcatacattttagaaacatcttagccgtaagttaggtcctgtgttaaa | reverse Primer |
| (also known as | ctgtttagtgctctgtttttaagaaaacaaatgttgaacctcacacttttatgtggtgacagt | tgcttgggtgaccttagg |
| CPSF100; | gtaatttaattaaaaggtgtaaatgttttcatctcttaggcttgctgtctcctaaggtcaccc | ag (SEQ ID NO: |
| KIAA1367; | aagcagtggttggattttatacacattactac (SEQ ID NO: 7) | 9) |
| CPSF2) | | Product size |
| | | 156 bp |
| CCRL2 | ctctcctgtatgcgtttcttgatgggacatttagcaaatacctctgccgctgtttccatctg | forward Primer |
| chemokine (C-C | cgtagtaacaccccacttcaacccagggggcagtctgcacaaggcacatcgaggga | ggcgcggaaatttgtct |
| motif) receptor- | agaacctgaccattccaccgaagtgtaaactagcatccaccaaatgcaagaagaata | aag (SEQ ID NO: |
| like 2 | aacatggattttcatctttctgcattatttcatgtaaattttctacacatttgtatacaaaatcg | 11) |
| (also known as | gatacaggaagaaaagggagaggtgagctaacatttgctaagcactgaatttgtctca | reverse Primer |
| HCR; CKRX; | ggcaccgtgcaaggctctttacaaacgtgagctccttcgcctcctaccacttgtccata | ccagggtttggagtttga |
| CRAM-A; | gtgtggataggactagtctcatttctctgagaagaaaactaaggcgcggaaatttgtcta | tg (SEQ ID NO: |
| CRAM-B; 3; | agatcacataactaggaagtggcagaactgattctccagccctggtagcatttgctcag | 12) |
| FLJ55815; M) | agcctacgcttggtccagaacatcaaactccaaaccctggggacaaacgacatga | Product size |
| | (SEQ ID NO: 10) | 117 bp |
| TNFAIP6 | ggataccccattgtgaagccagggcccaactgtggatttggaaaaactggcattattg | forward Primer |
| tumor necrosis | attatggaatccgtctcaataggagtgaaagatgggatgcctattgctacaacccacac | ttgaagatgacccaggt |
| factor, | gcaaaggagtgtggtggcgtctttacagatccaaagcaaatttttaaatctccaggcttc | tgc (SEQ ID NO: |
| alpha-induced | ccaaatgagtacgaagataaccaaatctgctactggcacattagactcaagtatggtca | 14) |
| protein 6 | gcgtattcacctgagttttttagattttgaccttgaagatgacccaggttgcttggctgatt | reverse Primer |
| (Also known as | atgttgaaatatatgacagttacgatgatgtccatggctttgtgggaagatactgtggag | catctggaagctcatctc |
| TSG6; TSG-6) | atgagcttccagatgacatcatcagtacaggaaatgtcatgaccttgaagtttctaagtg | cac (SEQ ID NO: |
| | atgcttcagtgacagctggaggtttccaaatcaaatatgttgcaatggatcctgtatcca | 15) |
| | aatccagtcaag (SEQ ID NO: 13) | Product size |
| | | 105 bp |
| CD44 | ctggatcaccgacagcacagacagaatccctgctaccagagaccaagacacattcca | forward Primer |
| (also known as | ccccagtggggggtcccataccactcatgaatctgaatcagatggacactcacatgg | aaggtggagcaaacac |
| IN; LHR; | gagtcaagaaggtggagcaaacacaacctctggtcctataaggacaccccaaattcc | aacc (SEQ ID |
| MC56; MDU2; | agaatggctgatcatcttggcatccctcttggccttggctttgattcttgcagtttgcattg | NO: 17) |
| MDU3; MIC4; | cagtcaacagtcgaagaaggtgtgggcagaagaaaaagctagtgatcaacagtggc | reverse Primer |
| Pgp1) | aatggagctgtggaggacagaaagccaagtggactcaacggagaggccagcaagt | gctttttcttctgcccaca |
| | ctcaggaaatggtgcatttggtgaacaaggagtcgtcagaaactccagaccagtttat | c (SEQ ID NO: |
| | gacagctgatgagacaaggaacctgcagaatgtggacatgaagattggggtgtaaca | 18) |
| | cctacaccattatcttggaaagaaacaaccgttgtaaacataaccattacagggagctg | Product size |
| | ggacacttaacagatgcaatgtgctactgattgtttcattgcga (SEQ ID NO: | 150 bp |
| | 16) | |
| TMEM45A | tgtttctcaccatatgcttttgttggcattatgcagtaaccattgtcatcgttggaatgaatt | forward Primer |
| Transmembrane | atgctttcattacctggttggttaaatctagacttaagaggctctgctcctcagaagttgg | agttggatgcccacact |
| protein 45A | acttctgaaaaatgctgaacgagaacaagaatcagaagaagaaatgtgactttgatga | atg (SEQ ID NO: |
| (also known as | gcttccagtttttctagataaaccttttcttttttacattgttcttggttttgtttctcgatctt | 20) |
| DERP7; | ttgtttggagaacagctggctaaggatgactctaagtgtactgtttgcatttccaatttggttaa | reverse Primer |
| FLJ10134) | agtatttgaatttaaatattttctttttagctttgaaaatattttgggtgatactttcattttgc | agtagcaagcccagta |
| | acatcatgcacatcatggtattcaggggctagagtgatttttttccagattatctaaagttgga | accttg (SEQ ID |
| | tgcccacactatgaaagaaatatttgttttatttgccttatagatatgctcaaggttactgg | NO. 21) |
| | gcttgctactatttgtaactccttgaccatggaattatacttgtttatcttgttgctgca | Product size |
| | (SEQ ID NO: 19) | 79 bp |
| WDR43 | gctcactaggggacatgcacatggaagctcacctctaagaaagggctgggcagatg | forward Primer |
| WDR43 HGNC | gatttattttttcccacctgtgaatatgtaaaacaaaaccattatctttgagggagtttttaat | acatgcacatggaagct |
| WD repeat | accantgacagaacagagatttgtgtgctcatcttaagagccagagccatataagcat | cac (SEQ ID NO: |
| domain 43 | cttgggaaagcaagtttgaaccagctgctggtgtaatgtacagttatatttgtctataaat | 23) |
| (also known as | ggagctgtttatggcaatttaataccattctctttgt (SEQ ID NO: 22) | reverse Primer |
| KIAA0007) | | aacttgctttcccaagat |
| | | gc (SEQ ID NO: |
| | | 24) |
| NBN NBN | agcgccccagtgaacactacaacatacgtagctgacacagaatcagagcaagcaga | forward Primer |
| HGNC nibrin | tacatgggatttgagtgaaaggccaaaagaaatcaaagtctccaaaatggaacaaaa | atagggcttctcagcag |
| (also known as | attcagaatgctttcacaagangcacccactgtaaaggagtcctgcaaaacaagctct | cag (SEQ ID NO: |
| ATV; NBS; | aataataatagtatggtatcaaatactttggctaagatgagaatcccaaactatcagcttt | 26) |
| P95; NBS1; AT- | caccaactaaattgccaagtataaataaaagtaaagatagggcttctcagcagcagca | reverse Primer |
| V1; AT-V2; | gaccaactccatcagaaactactttcagccgtctaccaaaaaaagggaaagggatga | tccacaatgagggtgta |
| FLJ10155; | agaaaatcaagaaatgtcttcatgcaaatcagcaagaatagaaacgtcttgttctctttta | gca (SEQ ID NO: |
| MGC87362) | gaacaaacacaacctgctacaccctcattgtggaaaaataaggagcagcatctatctg | 27) |
| | agaatgagcctgtggacacaaactcagacaataacttatttacagatacagatttaaaat | Product size |
| | ctattgtgaaaaattctgccagtaaatctcatgct (SEQ ID NO: 25) | 174 bp |
| ERMP1 | Gaagctttggtattgtacaaggtcagtagtaagatgctcactagtctcagggcttgtgta | forward Primer |
| endoplasmic | atattctgggaggtcatttaaatgccagaaatggtcaagcaattatacacagtatttatga | aatgccagaaatggtca |
| reticulum | ctctgttaagcataccgtttgtctgtcacattagtagattctgagattaaaaaaaatttttaa | agc (SEQ ID NO: |
| metallopeptidase | agagtgatcatttaaataatttctaaaagggtcttttcaagctctaacaaagtcactaaca | 29) |
| 1 | aatgcattattttctacagaattagatgttagtagtacagtactgcatattcagggaaaaa | reverse Primer |
| (also known as | gtgtgaggaattgatttcaaaatagttcgttcttgtgtttgacctaagaatgattgtcgcat | ttcctcacactttttccct |
| FXNA; | gaagtgtttgtttttacagtttagcatatataaacaaacatgataggattccttaagatgtta | ga (SEQ ID NO: |
| KIAA1815; | ccacccagggggccacaagccagcctgctgtctcaggaagctgtagaaggagtgttt | 30) |
| bA207C16.3) | gtcaatttcttgtcactggtttgctgacttactgaggattaattgttgccttacaatgttactg | Product size |
| | a (SEQ ID NO: 28) | 236 bp |
| LRP11 low | gtatggtgcctatctacactcacatgatattcttattcacgttttttttnaaccataagtggc | forward Primer |
| density | aaatattttaaaatatttgaaaaacactccagaatctagtacgctttatttttagactgaacc | ttcgggtggggaatata |
| lipoprotein | taaagtaggttgttcttttaacaaagggtttaattcgggtggggaatataacatatcaaaa | aca (SEQ ID NO: |
| receptor-related | tacatgaacaaatggaaagttacttctagaaaagcaaagaaattgggtatcatttttgttt | 32) |
| protein 11 | cttgggaagctaattttgttgaatgtttagaattgagcaaagatgtaaatttttgaagggc | reverse Primer |
| (also known as | agtttagaaaaattaactttgtgaatgaacttaagatgtctgtactctatatgtgatgctgtg | acaaactgcacagcatc |
| MANSC3; | cagtttgtttttatatggaaagatgtcaactatagccataaccaataaaataaaaactgat | aca (SEQ ID NO: |
| FLJ14735; | gaggcatgcagctttcagcacatcttttatacatgaagaaattaattttgtgttgctatggt | 33) |
| MGC39092; | gttgaaatatccaagatgttctgta (SEQ ID NO: 31) | Product size |
| DA350J20.3) | | 221 bp |
| CDKN2i cyclin- | | Product size |
| dependent | | 182 bp |
| kinase inhibitor | | |
| 2A (melanoma, | | |
| p16, inhibits | | |
| CDK4) | | |
| CDKN2A | | |
| HPV16E4 | caccgaagaaacacagacgactatccagcgaccaagatcagagccagacaccgga | forward Primer |
| Human | aacccctgccacaccactaagttgttgcacagagactcagtggacagtgctccaa | caccgaagaaacacag |
| Papillomavirus | (SEQ ID NO: 34) | acga (SEQ ID |
| Type 16 genome | | NO: 35) |
| | | reverse Primer |
| | | ttggagcactgtccactg |
| | | ag (SEQ ID NO. |
| | | 36) |
| HPV16E7 | gacaagcagaaccggacagagcccattacaatattgtaaccttttgttg | forward Primer |
| Human | caagtgtgactctacg | gacaagcagaaccgga |
| Papillomavirus | cttcggt (SEQ ID NO: 37) | caga (SEQ ID |
| Type 16 genome | | NO: 38) |
| | | reverse Primer |
| | | accgaagcgtagagtc |
| | | acac (SEQ ID |
| | | NO: 39) |
| SERP1 stress- | gaggaggcaggcgagtgagcgagtccgaggggtggccgggg | forward Primer |
| associated | caggtggtggcgccgcgaa | tctgtaggaccctggtta |
| endoplasmic | gatggtcgccaagcaaaggatccgtatggccaacgagaagcacagcaagaacatca | ttgg (SEQ ID NO: |
| reticulum | ccca | 41) |
| protein 1 | gcgcggcaacgtcgccaagacctcgagaaatgcccccgaagagaaggcgtctgta | reverse Primer |
| (also known as | ggacc | tcacatgcccatcctgat |
| RAMP4; | ctggttattggctctcttcatttttgttgtctgtggttctgcaattttccagattattca | ac (SEQ ID NO: |
| MGC117327; | aagtatcaggatgggcatgtgaagtgactgaccttaagatgtttccattctcctgtgaat | 42) |
| MGC133321; | ttttaacttgaactcattcctgatgtttgataccctggttgaaaacaattcagtaaagca | Product size |
| MGC133322) | tcctgcctcagaatgactttcctatcatgcttcatgtgtcattccaaggtttcttcatga | 93 bp |
| | gtcattccaagttttctagtcca (SEQ ID NO: 40) | |
| ASAH1 N- | gagggatgagacagacattcacctgtatatttcttttaatgggcacaaaatgggcccttg | forward Primer |
| acylsphingosine | cctctaaatannnntnnnnngnnttnaagaagtaatcagtatgcaaagcaatcttttat | cccagtaaccctaagga |
| amidohydrolase | a | agttg (SEQ ID |
| (acid | caataattgaagtgttnccntttttcataattactctacttcccagtaaccctaaggaag | NO: 44) |
| ceramidase) 1 | ttgctaacttaaaaaanctgcatcccacgttctgttaatttagtaaataaacaagtcaaa | reverse Primer |
| (also known as | gacttgtggaaaataggaagtgaacccatattttaaattctcataagtagcattcatgta | gggttcacttcctattttc |
| MGC173782; | ataaacaggtttttagtttgttcttcagattgatagggagttttaaagaaattttagtag | cac (SEQ ID NO: |
| zgc: 101637) | ttactaaaattatgttactgtatttttcagaaatcnaactgcttatgaaaagtactaata | 45) |
| | gaacttgttaacctttctaaccttcacgattaactgtgaaatgtacgtcatttgtgcaag | Product size |
| | accgtttgtccacttcattttgtataatcacagttgtgttcctgacactca (SEQ ID | 106 bp |
| | NO: 43) | |
| HIGD1A HIG1 | ttaattcaactttcgtctgcctgttttgtggactggctggctctgttagaactctgtcca | forward Primer |
| domain family, | aaaagtgcatggaatataacttgtaaagcttcccacaattgacaatatatatgcatgtgt | cttatgagcaagctggtt |
| member 1A | ttaaaccaaatccagaaagcttaaacaatagagctgcataatagtatttattaaagaatc | tgg (SEQ ID NO: |
| (also known as | accaactgtaaacatgagaataacttaaggttctagtttagttttttgtaattgcaaatt | 47) |
| HIG1; DKFZp56 | atatttttgctgctgatatattagaataattttaaaatgtcatcttgaaatagaaatatg | reverse Primer |
| 4K247) | tattttaagcactcacgcaaaggtaaatgaacacgttttaaatgtgtgtgttgctaattt | caccatggaatgcaaat |
| | tttccataagaattgtaaacattgaactgaacaaattacctataatggatttggttaatg | tcag (SEQ ID |
| | acttatgagcaagctggtttggccagacagtatacccaaacttttatataatatacagaa | NO: 48) |
| | ggctatcacacttgtgaaattctcttgtctaatctgaatttgcattccatggtgttaaca | Product size |
| | (SEQ ID NO: 46) | 113 bp |
| PGK1 | ctgtgggggtatttgaatgggaagcttttgcccggggaaccaaagctctcatg | forward Primer |
| Phosphoglycerat | gatgaggtggtgaa | ctgtgggggtatttgaat |
| kinase 1 | agccacttctaggggctgcatcaccatcataggtggtggagacactgc | gg (SEQ ID NO: |
| (also known as | cacttgctgtgccaaa | 50) |
| PGKA; MIG10; | tggaacacggaggataaagtcagccatgtgagcactgggggtggtgccag | reverse Primer |
| MGC8947; | tttggagctcctggaag (SEQ ID NO: 49) | cttccaggagctccaaa |
| MGC117307; | | ctg (SEQ ID NO: |
| MGC142128) | | 51) |

It was possible for the inventors, in particular for the markers listed in Table 2, the sequences of which are likewise indicated in the table, to investigate and detect their suitability for determination of the prediction of the predisposition of a person to the development of cervical cancer and/or cancer precursors. In our own experiments, the inventors showed that it is in particular possible by means of these markers to achieve a high positive prediction value of the diseases.

Presently, “diagnostic marker” or “diagnostic species” is understood here as meaning any diagnostic gene made available or the protein encoded by this gene as well as the RNA transcribed starting from the gene, or alternatively any sequence fragment characteristic for the gene or the protein or the mRNA. “Characteristic sequence fragment” is understood here as meaning any sequence section of the gene, of the protein encoded thereby or of the mRNA transcribed by the gene, which is specific for the respective gene, protein or mRNA, i.e. is to be found in this sequence, not in other genes, proteins or mRNAs.

Accordingly, in one embodiment of the method according to the invention it is preferred if the prediction is carried out by the determination of the gene expression of the diagnostic markers.

“Gene expression” is understood here, as similarly explained further above, as meaning the mRNA derived/transcribed by the marker genes, and the proteins in turn encoded by the marker genes. These are therefore presently also designated as “marker mRNA” and “marker proteins”.

The detection of the gene expression of the diagnostic markers offers the advantage that in the investigation of a human sample a comparison can thus be drawn to the gene expression of the corresponding markers in a healthy or a non-progressive sample. By means of the comparison, an over- or an under-regulation of these marker genes can optionally then be determined for the individual marker genes, according to which in this case the predisposition for the development of cervical cancer can be predicted.

Accordingly, in a first embodiment of the method according to the invention, it is preferred if the prediction is carried out by the detection of mRNA transcripts of at least one of the diagnostic markers, and in particular if the detection of the mRNA transcripts is carried out by means of a quantitative RT-PCR (real-time polymerase chain reaction).

Real-time quantitative PCR (presently also qRT-PCR for short) is a method for the amplification/duplication of nucleic acids, which is based on the principle of the conventional polymerase chain reaction (PCR), and additionally makes possible the quantification of the DNA obtained thereby. Quantification is generally carried out with the aid of fluorescence measurements that are recorded during a PCR cycle, the fluorescence increasing proportionally with the amount of PCR products. At the end of a course consisting of several cycles, quantification in the exponential phase of the PCR is performed with the aid of fluorescence signals obtained.

Quantitative real-time PCR is thus an excellent tool for the quantification of nucleic acids.

In preferred exemplary embodiments, the mRNA of the diagnostic markers is thus transcribed to cDNA and then used for quantitative real-time PCR, whereby the transcripts of the diagnostic markers are quantified and, based thereon, a prediction of a predisposition for the development of cervical cancer can be made.

It is in particular preferred here if sequences and/or sequence sections of the diagnostic markers are detected, which represent characteristic fragments and/or sections for the respective diagnostic marker, that is to say sections which are only to be found or to be detected in the respective marker quite specifically in the given sequence.

It is in particular preferred here if in, the quantitative real-time PCR, at least one oligonucleotide, preferably an oligonucleotide pair, is used that is selected from at least one of the following:
(a) an oligonucleotide/oligonucleotide pair or its complementary strand listed in Table 3 below;
(b) oligonucleotides that hybridize under stringent conditions to the oligonucleotides defined in (a), or fragments thereof; and
(c) oligonucleotides that have a sequence homology of at least 80% to the oligonucleotides from Table 3, and that are suitable as the oligonucleotides from (a) for the detection of the diagnostic markers disclosed herein.

TABLE 3
Oligonucleotides/primers used

| Primer | Marker |
|---|---|
| forward Primer | SERPINB5 |
| gttgccggttcatggattac (SEQ ID NO: 2) | serpin peptidase inhibitor, clade B |
| reverse Primer | (also known as PI5; maspin) |
| gcatgtcaaggaagagatgg (SEQ ID NO: | |
| 3) | |
| forward Primer | CAND1 |
| gcttgattttgaatgttcagatg (SEQ ID NO: | cullin-associated and neddylation-dissociated 1 |
| 5) | (also known as TIP120; T1P120A; FLJ10114; FLJ10929; |
| reverse Primer | FLJC) |
| gattatcacctgccacattttg (SEQ ID NO: | |
| 6) | |
| forward Primer | CPSF2 |
| gccgtaagttaggtcctgtgtt (SEQ ID NO. | cleavage and polyadenylation specific factor 2, 10OkDa |
| 8) | (also known as CPSF100; KIAA1367; CPSF2) |
| reverse Primer | |
| tgcttgggtgaccttaggag (SEQ ID NO: | |
| 9) | |
| forward Primer | CCRL2 |
| ggcgcggaaatttgtctaag (SEQ ID NO: | chemokine (C-C motif) receptor-like 2 |
| 11) | (also known as HCR; CKRX; CRAM-A; CRAM-B; 3; |
| reverse Primer | FLJ55815; M) |
| ccagggtttggagtttgatg (SEQ ID NO: | |
| 12) | |
| forward Primer | TNFAIP6 |
| ttgaagatgacccaggttgc (SEQ ID NO: | tumor necrosis factor, |
| 14) | alpha-induced protein 6 |
| reverse Primer | (Also known as TSG6; TSG-6) |
| catctggaagctcatctccac (SEQ ID NO: | |
| 15) | |
| forward Primer | CD44 |
| aaggtggagcaaacacaacc (SEQ ID NO: | (also known as IN; LHR; MC56; MDU2; MDU3; MIC4; |
| 17) | Pgp1) |
| reverse Primer | |
| gctttttcttctgcccacac (SEQ ID NO: | |
| 18) | |
| forward Primer | TMEM45A Transmembranprotein 45A |
| agttggatgcccacactatg (SEQ ID NO: | (also known as DERP7; FLJ10134) |
| 20) | |
| reverse Primer | |
| agtagcaagcccagtaaccttg (SEQ ID | |
| NO: 21) | |
| forward Primer | WDR43 WDR43 HGNC WD repeat |
| acatgcacatggaagctcac (SEQ ID NO: | domain 43 |
| 23) | (also known as KIAA0007) |
| reverse Primer | |
| aacttgctttcccaagatgc (SEQ ID NO: | |
| 24) | |
| forward Primer | NBN NBN HGNC nibrin |
| atagggcttctcagcagcag (SEQ ID NO: | (also known as ATV; NBS; P95; NBS1; AT-V1; AT-V2; |
| 26) | FLJ10155; MGC87362) |
| reverse Primer | |
| tccacaatgagggtgtagca (SEQ ID NO: | |
| 27) | |
| forward Primer | ERMP1 endoplasmic |
| aatgccagaaatggtcaagc (SEQ ID NO. | reticulum metallopeptidase 1 |
| 29) | (also known as FXNA; KIAA1815; bA207C16.3) |
| reverse Primer | |
| ttcctcacactttttccctga (SEQ ID NO: | |
| 30) | |
| forward Primer | LRP11 low density |
| ttcgggtggggaatataaca (SEQ ID NO: | lipoprotein receptor-related |
| 32) | protein 11 |
| reverse Primer | (also known as MANSC3; FLJ14735; |
| acaaactgcacagcatcaca (SEQ ID NO: | MGC39092; DA350J20.3) |
| 33) | |
| forward Primer | HPV16E4 Human Papillomavirus |
| caccgaagaaacacagacga (SEQ ID NO: | Type 16 genome |
| 35) | |
| reverse Primer | |
| ttggagcactgtccactgag (SEQ ID NO: | |
| 36) | |
| forward Primer | HPV16E7 Human Papillomavirus |
| gacaagcagaaccggacaga (SEQ ID NO: | Type 16 genome |
| 38) | |
| reverse Primer | |
| accgaagcgtagagtcacac (SEQ ID NO: | |
| 39) | |
| forward Primer | SERP1 stress-associated |
| tctgtaggaccctggttattgg (SEQ ID NO: | endoplasmic reticulum protein 1 |
| 41) | (also known as RAMP4; MGC117327; |
| reverse Primer | MGC133321; MGC133322) |
| tcacatgcccatcctgatac (SEQ ID NO: | |
| 42) | |
| forward Primer | ASAH1 N-acylsphingosine |
| cccagtaaccctaaggaagttg (SEQ ID | amidohydrolase (acid |
| NO: 44) | ceramidase) 1 |
| reverse Primer | (also known as MGC173782; zgc: 101637) |
| gggttcacttcctattttccac (SEQ ID NO: | |
| 45) | |
| forward Primer | HIGD1A HIG1 domain family, member 1A |
| cttatgagcaagctggtttgg (SEQ ID NO: | (also known as HIG1; DKFZp564K247) |
| 47) | |
| reverse Primer | |
| caccatggaatgcaaattcag (SEQ ID NO: | |
| 48) | |
| forward Primer | PGK1 Phosphoglyceratkinase 1 |
| ctgtgggggtatttgaatgg (SEQ ID NO: | (also known as PGKA; MIG10; MGC8947; |
| 50) | MGC117307; MGC142128) |
| reverse Primer | |
| cttccaggagctccaaactg (SEQ ID NO: | |
| 51) | |

“Oligonucleotide/s” are presently understood as meaning—as also in the prior art and in the field concerned generally—isolated or purified oligonucleotides or oligomers synthetically or recombinantly constructed from a few nucleotides (DNA or RNA), whereby the nucleotide sequence generally consists of about 10 to 100 nucleotide units.

Presently, the oligonucleotides are also used in the polymerase chain reaction, which is why the oligonucleotides claimed and disclosed are also designated as “primers”. In addition, the general designation of specific oligonucleotides by “forward” or “reverse” reproduced in Table 3 are also customary designations of primers/oligonucleotides in the relevant technical field in Germany so that presently these English terms are also chosen in order to provide clarity.

“Hybridization under stringent conditions” is presently understood as meaning that the hybridization is carried out in vitro under conditions that are stringent enough in order to guarantee a specific hybridization. Such stringent hybridization conditions are known to the person skilled in the art and can be inferred, for example, from the literature (see Sambrook et al. (2001), Molecular Cloning: A Laboratory Manual, 3rd edition, Cold Spring Harbor Laboratory Press, Cold Spring Harbor, N.Y.). Generally, presently “specifically hybridize” means that a molecule, presently in particular an oligonucleotide, preferentially binds to a specific nucleotide sequence under stringent conditions if this sequence is present in a complex mixture of DNA or RNA, and thus not, or to a markedly lower extent, to other sequences. Stringent conditions are, inter alia, sequence-dependent and will be different under different circumstances. Longer (oligonucleotide) sequences specifically hybridize at higher temperatures. In general, stringent conditions are selected such that the temperature is approximately 5° C. below the thermal melting point (Tm) for the specific oligonucleotide sequence. The Tm is the temperature (under defined ionic strength, pH and nucleic acid concentration) at which 50% of the molecules complementary to the target sequence hybridize to the target sequence in the equilibrium state. Typically, stringent conditions are those in which the salt concentration is at least approximately 0.01 to 1.0 M sodium ions concentration (or another salt) at a pH between 7.0 and 8.3 and the temperature is at least 30° C. for shorter molecules (that is, for example, 10-50 nucleotides). Stringent conditions can additionally be achieved by addition of destabilizing agents, such as, for example, formamide.

Thus the hybridization can take place, for example, under the following conditions: hybridization buffer: 2×SSC, 10× Denhart's solution (Ficoll 400+PEG+BSA; ratio 1:1:1), 0.1% SDS, 5 mM EDTA, 50 mM Na₂HPO₄, 25011 g/ml of herring sperm DNA; 50 μg/ml of tRNA or 0.25 M sodium phosphate buffer pH 7.2, 1 mM EDTA, 7% SDS at a hybridization temperature of 65° C. to 68° C., wash buffer: 0.2×SSC, 0.1% SDS at a wash temperature of 65° C. to 68° C.

The oligonucleotides listed under c) have a sequence identity of at least 80%, preferably of at least 90% and most preferably at least 95% to the oligonucleotides indicated under a) or parts thereof, based on the sequences of the oligonucleotides indicated under a) shown in Table 3.

Preferably, the sequence identity of oligonucleotides according to c) is determined by comparison with the sequences indicated in Table 1. If two nucleic acid sequences of different length are compared with one another, the sequence identity preferably relates to the percentage proportion of the nucleotide radicals of the shorter sequence that are identical with the corresponding nucleotide radicals of the longer sequence. Sequence identities are customarily determined by means of various alignment programs, such as, for example, CLUSTAL. Generally, suitable algorithms are available to the person skilled in the art for the determination of the sequence identity, e.g. also the program that is publicly accessible and permanently accessible under http://www.ncbi.nlm.nih.gov/BLAST (e.g. the link “Standard nucleotide-nucleotide BLAST”).

In another embodiment of the diagnostic markers according to the invention, it is preferred if the prediction is carried out by the detection of the proteins encoded by the diagnostic markers.

It is understood that this detection of the proteins encoded by the diagnostic markers does not always have to take place here for the entire protein, but can also take place by means of certain, in each case specific, sections. The proteins can be detected, for example, by means of specific antibodies, i.e. antibodies that specifically bind to the proteins/protein fragments or sections, e.g. using Western blots and enzyme-coupled immunoabsorption assays. Methods for the quantitative detection of proteins are adequately known in the prior art, with respect to this reference is made, for example, to “Using Antibodies: A Laboratory Manual. Ed Harlow/David Lane 1999, Cold Spring Harbor Laboratory Press”, which is a suitable textbook and contains instructions for the methods described.

In a preferred embodiment, only one diagnostic marker is used in the method for the prediction of the predisposition of a person to the development of cervical cancer, whereas in other embodiments it is preferred if two or more of the diagnostic markers listed in Table 1 or 2 are used in combination for the prediction.

In the method according to the invention, it is preferred if the prediction is carried out in comparison to at least one of the following markers:
a) markers corresponding to the marker(s) to be tested from a suitable HPV-negative human sample;
b) markers corresponding to the tested marker(s) from a HPV-positive, non-progressive human sample; and/or;
c) reference markers that occur at a constant level in various human tissue/fluids.

By “markers corresponding to the marker(s) to be tested” it is presently meant that, if in a sample to be investigated, for example, the marker TMEM45A (or one of the other markers according to the invention disclosed herein) is to be used in the method for the prediction to predisposition, then in a corresponding (known) sample, which is detectably HPV-negative or HPV-positive, but at least non-progressive, the marker TMEM45A, in particular its gene expression, is likewise determined.

In particular, it is preferred if the prediction is carried out by the detection of a change, in particular a down- and/or up-regulation, of the gene expression of the diagnostic markers in comparison to the gene expression of at least one of the markers a) to c):
a) markers corresponding to the diagnostic marker(s) tested from a suitable HPV-negative human sample;
b) markers corresponding to the diagnostic marker(s) tested from an HPV-positive, non-progressive human sample; and/or;
c) reference markers that occur at a constant level in various human tissue/fluids.

The inventors have demonstrated in their own experiments that on the one hand certain marker genes are either up-regulated or down-regulated in their gene expression in comparison to the corresponding markers from an HPV-negative or HPV-positive, but non-progressive sample, and that this different gene expression of the markers can be used in the method for the prediction of the predisposition of a human for the development of cervical cancer.

In the method according to the invention, it is preferred if the step of detection is carried out by means of the determination of the gene expression of the diagnostic markers in the isolated sample, and in particular if it is carried out by means of the determination of the transcripts of the diagnostic markers and/or of the proteins encoded by the diagnostic markers. It is preferred here if, for the detection of the diagnostic markers, an oligonucleotide is used that is selected from the sequences listed in Table 3.

The term definitions and advantages shown further above for the use according to the invention also apply for the method according to the invention.

In a preferred embodiment, the method according to the invention contains the following steps:
(a) isolation of mRNA from a sample obtained from a person or subject:
(b) transcription of the mRNA isolated in step (a) to cDNA;
(c) bringing the cDNA sample obtained in step b) into contact with at least one oligonucleotide that is selected from the oligonucleotides listed in Table 3;
(d) carrying out a quantitative real-time PCR for the preparation of amplification products of the diagnostic markers transcribed to cDNA; and
(e) determination of the amplification products obtained in step (d).

Here, it is preferred in a refinement if the method contains the additional step (f):
(f) comparison of the amplification products determined in step (e) with amplification products corresponding to these from corresponding comparison markers from an HPV-negative suitable human sample and/or a suitable HPV-positive, but non-progressive human sample, whereby a change, in particular a down- and/or up-regulation, of the gene expression of the diagnostic markers in comparison to the comparison markers shows the predisposition of a human to develop cervical cancer.

The isolation of the mRNA can be carried out here using agents known in the prior art, for example by means of the RNeasy® mini-kits of the company Qiagen, Hilden, Germany. The transcription of the mRNA thus isolated can likewise be carried out using agents known in the prior art, for example by means of reverse transcriptase kits of the company Qiagen. It is understood that other agents and methods from other companies are also suitable for use in the method according to the invention; reference is made, with respect to these, to the standard work of Sambrook and Maniatis (Molecular Cloning: A Laboratory Manual; January 2001 edition).

In the method according to the invention, it is otherwise preferred, as also in the method according to the invention, if reference markers are detected in parallel to the detection of the diagnostic markers.

Here, presently, “reference markers” is intended to mean any diagnostic marker or any human gene that occurs ubiquitously and is expressed at a constant level in various genes, the expression of the genes remaining constant under various conditions.

Presently, for the method according to the invention as well as for the method, in particular at least one of the following reference genes, or their gene expression products, are preferred that have the following official gene names: ASAH1 (N-acylsphingosine amide hydrolase 1, HIGD1A (HIG1 domain family member 1A; hypoxia-inducible gene 1), SERP1 (stress-associated endoplasmic reticulum protein 1), PGK1 (phosphoglycerate kinase).

By means of the reference genes, for example, the transcription patterns of the mRNA can be standardized between various samples, and thus the relative amounts obtained then compared with one another.

In the method according to the invention, it is further preferred if the sample to be investigated, isolated from a subject, is a tissue or body fluid sample, and in particular a biopsy, a cervical cell sample and/or a Papanicolaou smear of a human.

In the case of the isolated or purified, synthetic or recombinant oligonucleotides according to the invention, it is preferred if they are selected from at least one of the following:
(a) an oligonucleotide listed in Table 3 or its complementary strand;
(b) oligonucleotides that hybridize under stringent conditions to the oligonucleotides defined in (a), or fragments thereof; and
(c) oligonucleotides that have a sequence homology of at least 80%, preferably of at least 85%, 90%, 95% or 98% to the oligonucleotides defined in (a) and with which the diagnostic markers can likewise be detected.

The intended purposes, definitions and means for detection explained further above for the method according to the invention apply to the oligonucleotides listed under the alternatives a), b) and c), their intended purpose or definition and/or detection.

The kit according to the invention for carrying out the method according to the invention here contains at least one oligonucleotide according to the invention, preferably an oligonucleotide pair listed in Table 3, or the oligonucleotide pairs listed in Table 3, which hybridize under stringent conditions to the oligonucleotide pairs listed in Table 3, or fragments thereof.

It is understood that besides the at least one oligonucleotide or the oligonucleotide pair further reagents and/or substances known in the prior art for the method according to the invention can be contained, consequently thus agents with which the method according to the invention can be carried out. Such agents, reagents and substances are in particular means for carrying out a polymerase chain reaction, that is, for example, the buffers and enzymes or nucleotides necessary for this. It will be clear to the person skilled in the art which reagents must be contained in the kit together with the oligonucleotides according to the invention in order to be able to carry out the method according to the invention successfully.

The intended purposes, definitions and means for detection explained further above for the method according to the invention also apply for the oligonucleotides/oligonucleotide pairs present in the kit according to the invention.

### SEQUENCE LISTING

The Sequence Listing is submitted as an ASCII text file (Sequence_Listing.txt, Oct. 18, 2013, 18.5 KB), which is incorporated by reference herein.

The kit can furthermore contain oligonucleotides for the detection of at least one reference marker, in particular at least one of the following: ASAH1 (N-acylsphingosine amide hydrolase 1, HIGD1A (HIG1 domain family member 1A; hypoxia-inducible gene 1), SERP1 (stress-associated endoplasmic reticulum protein 1), PGK1 (phosphoglycerate kinase) or gene expression products thereof.

The invention furthermore relates to the use of antibodies in the method, wherein the antibodies bind to proteins which are expressed by genes that are listed in Table 1, for the detection of a persistent infection with the human papilloma virus (HPV), in particular of an antibody directed against Temem45A, SerpinB5 or Cdkn2A.

It is understood that the features described above and those still to be explained below are not only usable in the combination indicated in each case, but also in other combinations or in isolation without departing from the scope of the present invention.

### BRIEF DESCRIPTION OF THE DRAWINGS

The invention is illustrated in more detail in the following description of the examples or exemplary embodiments below with the aid of the figures. These show the following:

FIG. 1 a summary of the cohort study used in the scope of the present invention

FIG. 2 a bar chart that shows graphically the correlation of microarray experiments in which changes in the gene expression were determined in samples, and quantitative real-time PCR experiments with which the change in the gene expression were likewise investigated in samples, and for the creation of which the results of the HPV-negative and HPV-positive samples were compared; and

FIG. 3 a bar chart that shows graphically the correlation of microarray experiments in which changes in the gene expression were determined in samples, and quantitative real-time PCR experiments with which the change in the gene expression were likewise investigated in samples, and for the creation of which results from the group of HPV-positive progressors and the HPV-positive non-progressors were compared.

FIG. 4 the statistical evaluation of the qPCR data of p16/CDKN2A, SERPINB5, TMEM45A, with PGK1 as a reference gene, whereby HPV-negative (N, n=19) was compared with HPV16 persistent (P, n=81) (Mann-Whitney test (two-sided));

FIG. 5 the ROC analysis of the qPCR data of p16/CDKN2A, SERPINB5, TMEM45A, whereby HPV-negative (N, n=19) was compared with HPV16 persistent (P, n=81) (Mann-Whitney test (two-sided));

FIG. 6 the statistical evaluation of the qPCR data of p16/CDKN2A, SERPINB5, TMEM45A, with PGK1 as a reference gene, whereby no progression (N, n=37) was compared with severe dysplasias (P, n=20) (Mann-Whitney test (two-sided));

FIG. 7 the ROC analysis of the qPCR data of p16/CDKN2A, SERPINB5, TMEM45A, whereby no progression (N, n=37) was compared with severe dysplasias (P, n=20) (Mann-Whitney test (two-sided)); and

FIG. 8 Immunohistochemical investigations on frozen sections of biopsy material (normal cervix or cervical carcinoma (HPV16-pos)), using TEMEM45A antibodies: P-18, Santa Cruz: A and C-13, Santa Cruz: B, SERPINB5/Maspin antibody: 554292, BD Pharmingen: C.

### DETAILED DESCRIPTION OF THE FIGURES AND OF PREFERRED EMBODIMENTS

### Materials and Methods:

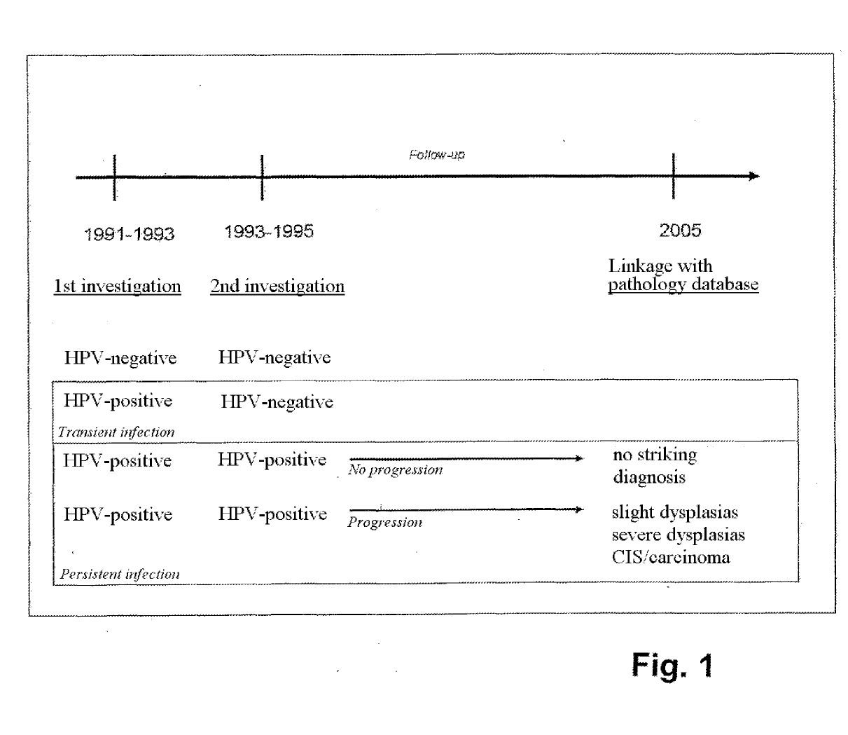

As already mentioned further above, the investigation material for the present invention originates from a Danish cohort study, for which women between 20 and 29 years were randomly selected. FIG. 1 shows a summary of the sample material used in the present application. Thus, in the years 1991 to 1993 samples of endo- and ectocervix were taken from about 11,088 Danish women, and Pap smears were performed; both samples were transferred to buffer and investigated as to whether these were HPV-negative or HPV-positive. In the years 1993 to 1995, the second investigation of 78% of the women investigated beforehand took place, and ten years later, in the year 2005, the linkage with the pathology database took place, after which the women were identified who developed slight to severe dysplasias/carcinomas.

FIG. 1 also illustrates schematically that a transient infection is to be understood as meaning HPV infections, which after a certain period of time (2 years) are no longer detectable and thus after the period of time as HPV infection is no longer present. (that is: HPV-positives in the first investigation were diagnosed as HPV-negatives in the second investigation). In these then, just as in HPV-negatives already diagnosed with the first investigation, no progression occurred. In contrast to this, the results which were HPV-positive in both investigations showed progression.

For the gene expression analysis by means of microarray, with which the gene expression of many genes can be determined simultaneously from a small amount of sample material, profiles of 52 samples (taken in the second phase) of the Danish cohort were determined and compared with one another. The analysis was diagnosed with respect to HPV-negatives against HPV-positives, HPV16-positives without progression, i.e. without noticeable cytological problems after the 10-year follow-up) against HPV16-positive progressors, i.e. during the 10-year follow-up, a slight dysplasia, a severe dysplasia, a carcinoma in situ (below also: CIS) or cancer/a carcinoma was diagnosed. The genes for which differential gene expression was detected are listed in the above Tables 1 and 2 with their respective gene names.

By the comparison of the groups, on the one hand genes were identified which in HPV-negative samples showed a different expression than the HPV16-positive sample, and on the other hand genes were identified whose expression was different between women who had no histological changes during the follow-up, and women who subsequently developed dysplasias and carcinomas.

In order to validate the knowledge about the differential gene expression obtained by means of the microarray analysis, the samples were investigated by means of quantitative real-time polymerase chain reaction. For this, so far the following samples were investigated: 19 samples HPV-negative (no dysplasia up to the second investigation), and a total of 81 samples HPV16-positive, of these 37 non-progressive (without dysplasia development) and 44 progressive, of which in turn 10 samples with slight dysplasias, 20 samples with severe dysplasias, 12 with CIS and 2 with cancer/carcinoma.

The total RNA of the samples frozen at −80° C. was isolated by means of a modified RNeasy mini kit (Quiagen, Hilden, Germany), for which the samples were thawed briefly and carefully.

The RNA thus isolated was then either transcribed directly to cDNA by means of the reverse transcription kit (Quiagen), or else first additionally amplified for the generation of cRNA, and then transcribed to cDNA.

For the quantitative real-time PCR, primers were designed that are specific for the individual genes. The primers finally developed accordingly fulfilled the following characteristics: a) the sequence to which the primer binds occurs only once in the human genome; b) the hybridizing sequence was 18 to 23 nucleotides long; c) the melting temperature was between 58° C. and 62° C. (optimum: 60° C.); the GC content of the complete sequence was between 30% and 80% (optimum: 50%).

The primers finally generated are listed in Table 2, right column, as well as in Table 3.

The quantitative real-time PCR was carried out in a LightCycler 480 (Roche, Mannheim, Germany), for which the specific primers and SYBER Green were used, which intercalates in double-stranded DNA. By this binding, the fluorescence emitted is amplified at identical stimulation intensity by a large amount. During the amplification, after each cycle the fluorescence is measured in the LightCycler at the end of the elongation phase. The respective batch for the real-time PCR was as follows:

20 μl batch: 10 μl of SybrGReen I master (Roche)
2 μl of forward primer (3 μM)
2 μl of reverse primer (3 μM)
1 μl of H₂O (DEPC-treated)

Subsequently, in each case 5 μl of the corresponding cDNA were added and the following program was carried out:

| | | |
|---|---|---|
| Initial activation step: | 95° C. | 5 min |
| Denaturation: | 94° C. | 15 sec |
| Primer binding: | 55° C. | 30 sec |
| Product amplification: | 72° C. | 30 sec |
| Melt curve: | 95° C. | 5 min |
| | 65° C. | 1 min |
| | 72° C. | |
| End | 4° C. | |

By means of the accompanying software (Roche), it was possible to determine the crossing point (CP) value, that is the cycle in which the signal intensity of the cDNA sample stands out against the background fluorescence. This CP value serves as an indirect indicator of the gene expression, i.e. samples with a high gene expression of a gene show lower CP values than a sample with lower gene expression.

Additionally to the genes which showed a different expression between the groups to be investigated in the microarrays, reference genes were also measured that showed almost no differences. By means of the reference genes, the investigated genes were normalized. Each PCR comprised at least one template control and two positive controls. For the comparison of the results from the microarray tests and the qRT-PCR, the n-fold expression was calculated with the aid of the following formula:

n-fold expression=(efficiency of target gene)^{ΔCP target gene(control sample)}/(efficiency of reference)^{ΔCP reference(control sample)}

### Results

In Table 2, in the left column, those names of the marker genes are listed whose expression was investigated by means of the qRT-PCR. In the second column is found the sequence of these genes, and in the third column the oligonucleotides employed for the amplification of a certain, characteristic or specific section of the marker genes. Furthermore, the size of the reaction product is also indicated in the third column.

The following markers, or genes, were selected:

Chemokine (C-C motif) receptor-like protein 2 (CCRL2), tumor necrosis factor, alpha-induced protein 6 (TNFAIP6), which were both decreased in the expression; CD44 antigen, KIAA1815 and the transmembrane protein 45A (TMEM45A), whose expression was completely induced. The above genes were identified in the comparison of HPV-positive samples to HPV-negative samples.

The following markers were further identified in the comparison of the HPV16-positive, non-progressive samples with the HPV16-positive, progressive samples:

Serpine peptidase inhibitor, member 5 (SERPINB5), cullin-associated and neddylation-dissociated protein 1 (CAND1), cleavage- and polyadenylation-specific factor 2 (CPSF2), splice factor 3b, subunit 3 (SF3B3), whose gene expression was completely induced, and SEC14-like protein 1 (SEC14L1), whose gene expression was decreased.

The following genes were employed as reference genes that were used for the normalization of the samples:

ASAH1 (N-acylsphingosine amide hydrolase 1, H1GD1A (HIG1 domain family member 1A; hypoxia-inducible gene 1), SERP1 (stress-associated endoplasmic reticulum protein 1), PGK1 (phosphoglycerate kinase), or gene expression products thereof.

Further information on the genes mentioned is, as already mentioned further above, to be found by means of their indicated abbreviations, for example, in the database of the National Center for Biotechnology Information (http://www.ncbi.nlm.nih.gov/sites/entrez) under the search tool “genes”.

Since many of the RNAs extracted from the samples showed a low concentration, a two-round amplification of the RNA for the generation of con cRNA was carried out for the microarrays and also for a test batch of the qRT-PCR in order to increase the amounts of RNA. The amplified RNA (and also non-amplified RNA) was transcribed to cDNA and used for the qRT-PCR.

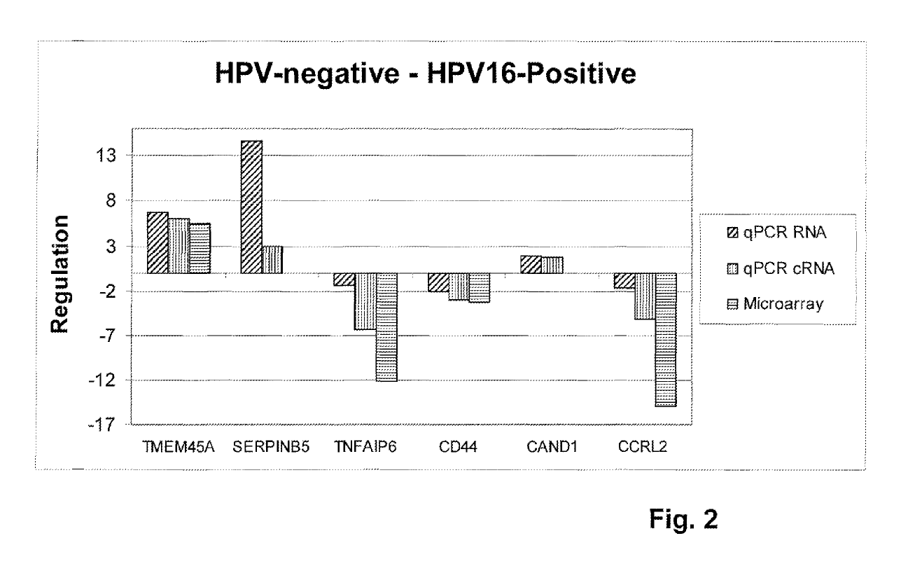

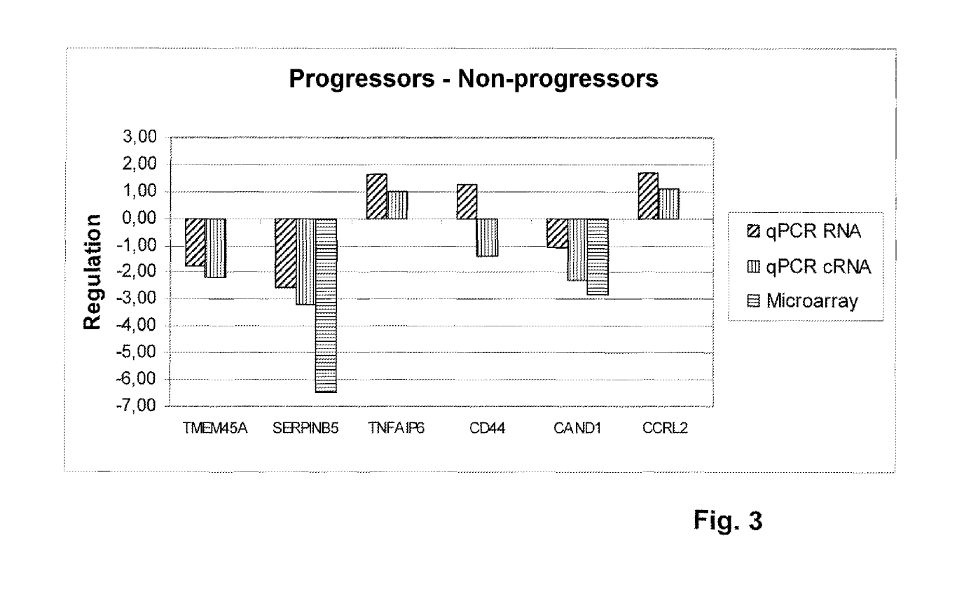

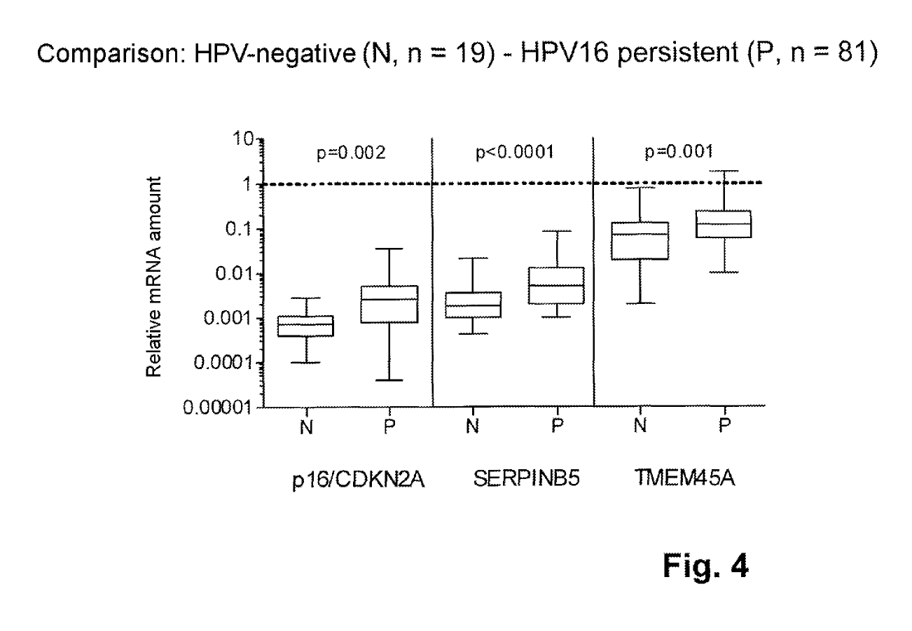

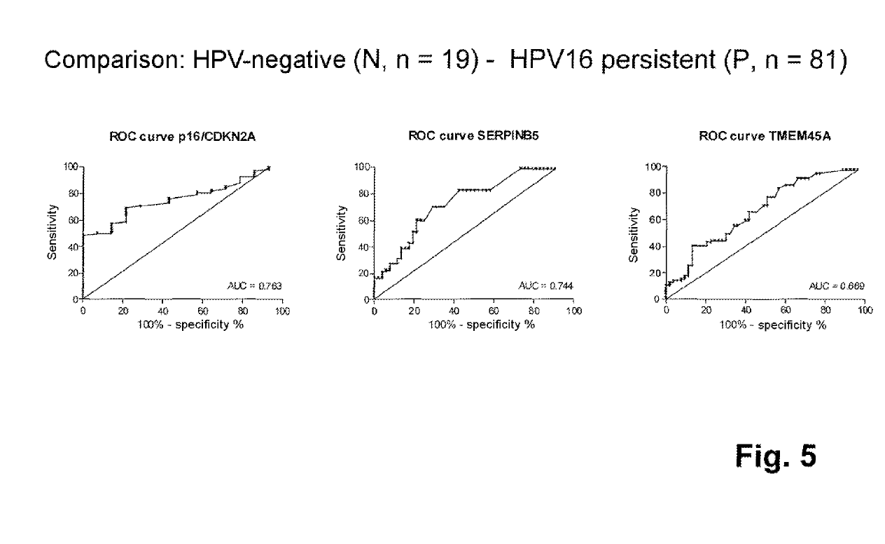

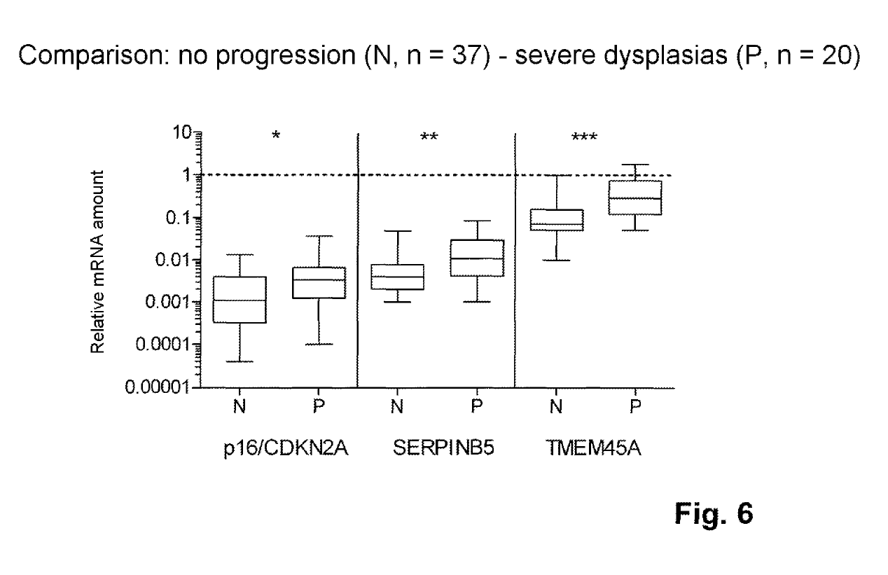

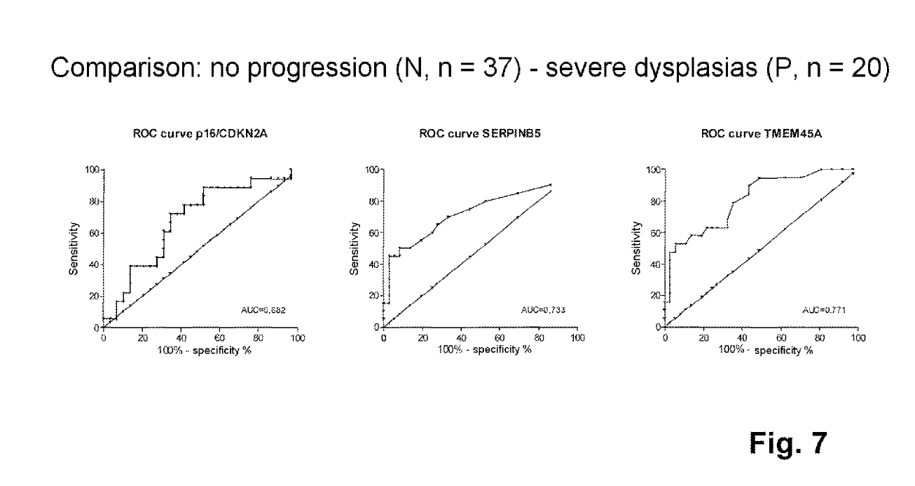

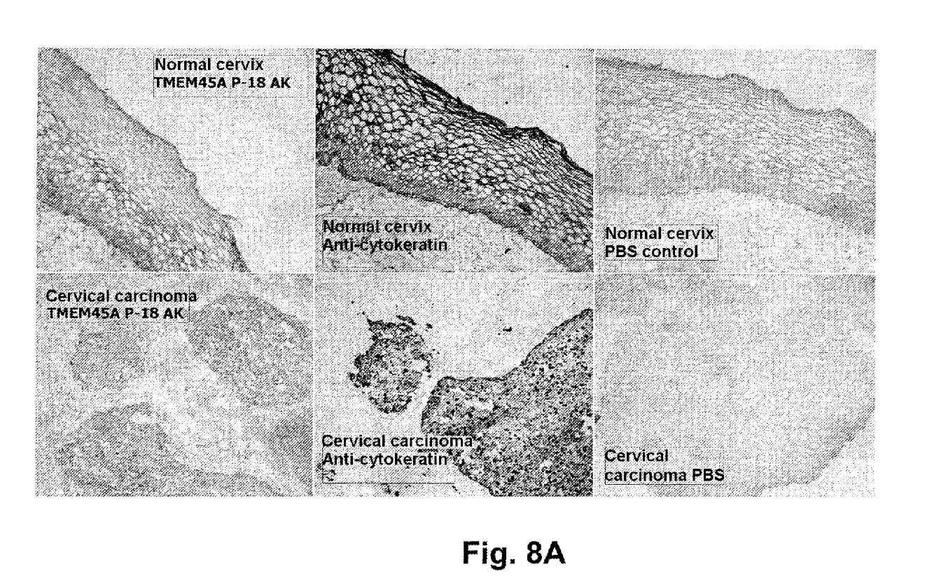

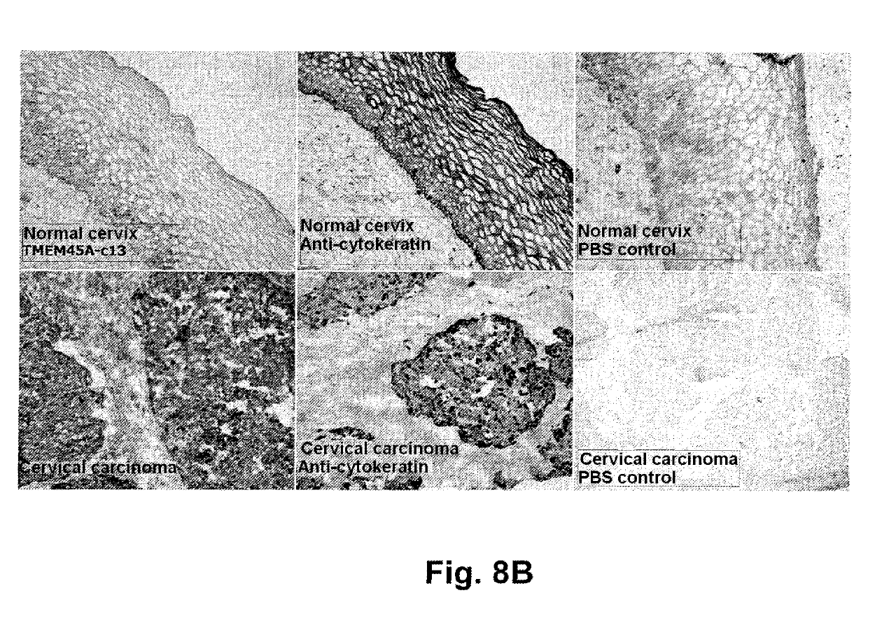

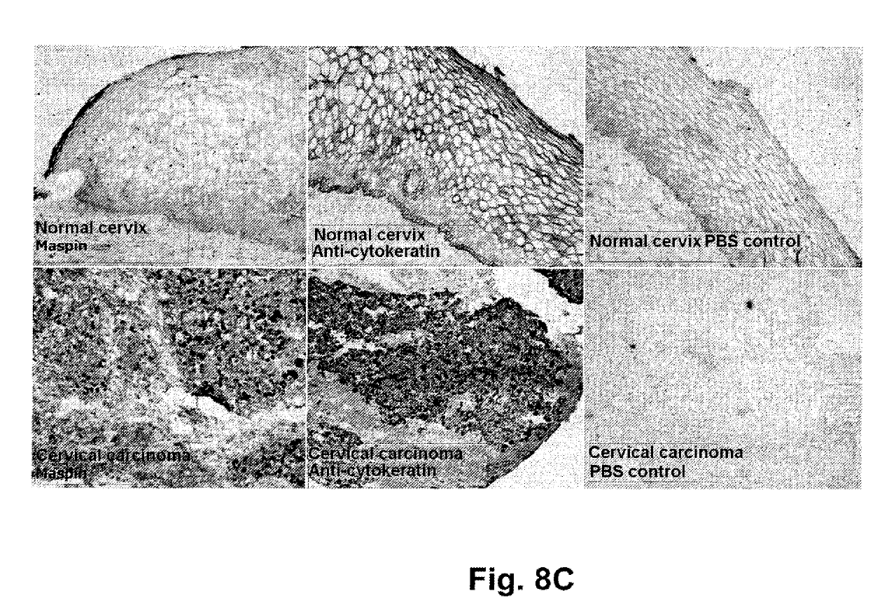

In order to be able to compare the n-fold expression of a gene from the microarray experiments with those of the qRT-PCR, the modified expression of the individual genes was calculated using an alternative method: For this the median of the CP values of the individual samples in a group was calculated. The median of the PGK1 values of the respective groups serves as a reference.
a) It was shown that those genes which had shown a different expression in the microarrays between HPV-negative and HPV-positive samples showed an approximately identical change of the expression in the qRT-PCR.
   The qPCR data of the cRNA were additionally tested for statistical relevance. It was seen that in the group of the HPV-negatives against the HPV16-positive samples all genes that were identified in the microarrays showed a significant expression difference, that is in all statistical tests p values <0.05 or in the case of TMEM45A even <0.000.
   The results of this statistical evaluation of the qPCR data for p16/CDKN2A, SERPINB5, TEMEM45A are shown in FIG. 4, whereby PGK1 was used as a reference gene. It was seen that all expression differences are highly significant.
   The correlation of data from microarray experiments with data from qRT-PCR experiments is shown in FIG. 2. As the bar diagram shown in FIG. 2 shows, the extent of the gene expression changes in qPCR experiments with cRNA, the original RNA and the microarray analyses with TMEM45A and CD44 correlates on comparison of HPV-negative with HPV16-positive samples. In the case of TNFAIP6 and CCRL2, a deregulation in the microarray analyses was observed, which it was not possible to confirm in the qPCR experiments of the original RNA. CAND1 is similarly strongly regulated in cRNA and original RNA, SERPINB5 is deregulated more strongly in the original RNA than in the cRNA.
   Furthermore, a so-called ROC analysis (“Receiver-Operator Characteristics”) of the qPCR data was carried out, in which analysis strategies are assessed and optimized. The ROC curve visually represents the dependence of the efficiency with the error rates for various parameter values. If the AUC (“Area under the curve”) value determined here is >0.5, this shows that there is no random distribution, but that the different expression of the genes is suitable as a test. It was possible to show this for p16/CDKN2A, SERPINB5 and TEMEM45A (see FIG. 5).
b) On quantitative comparison of the group of progressors with the non-progressors, a clear difference in expression was seen for SERPINB5 and TMEM45A (see FIG. 3). In contrast to microarray and cRNA analysis, CAND1 showed no deregulation in the original RNA. In accord with the significance analysis, it was not possible to observe any deregulation for CCRL2, CD44 or TNFAIP6.
   Other markers for the progression and persistence of HPV infections should be investigated in further experiments. Thus, in one experimental approach the amounts of E6/E7 transcripts were determined. For this, an E7-specific primer pair which detects the 5′-region of the viral RNA was detected and an E4-specific primer pair which can detect the 3′-range of the HPV16 transcripts was developed (see Table 2, left column and Table 3, right column)
   Additionally, the transcript amounts of CDKN2A, which codes for two structurally different proteins, p16 and p14ARF, as well as of SERPINB5 and TMEM45A, were investigated.
   In the original RNA, the relative mRNA amounts of HPV16E4, HPV16E7 and CDKN2A were determined by qPCR. Evaluation was carried out after normalization with PGK1. Statistical differences were calculated, as already calculated for the other experiments described further above, by means of the Wilcoxon test. The evaluation showed an expectedly high significance between HPV-positive and HPV-negative samples for the viral transcripts E4 and E7 (p=0.0002). A significant difference between these groups could also be determined for the expression of the CDKN2A gene (p=0.00029). A significant difference could also be determined between the two HPV-positive groups (progressors/non-progressors) for E4 or CDKN2A (p=0.02 and p=0.0053 respectively).
   FIG. 6 furthermore shows the results of the statistical evaluation of the qPCR data with PGK1 as the reference gene on comparison between no progression (in FIG. 6: N; n=37) and severe dysplasias (in FIG. 6: N; n=20). It is seen here that the expression differences are significant, inter alia, for CDKN2A, SERPINB5 and TMEM45A.
   In FIG. 7, the ROC analysis of the qPCR data of p16/CDKN2A, SERPINB5 and TMEM45A is shown analogously to FIG. 5, whereby in turn an AUC value >0.5 indicates that it is not a random distribution, but that the different expression of the genes is suitable as a test, which was the case, inter alia, with the three genes shown.
c) In the following, frozen section of biopsy material (normal cervix or cervical carcinoma (HPV16-positive) were prepared, and immunohistochemical investigations thus carried out. Here, the primary antibody (TEMEM45A antibody (P-18, Santa Cruz, sc100197 and C-13, Santa Cruz, sc100196); SERPIN/Maspin antibody (554292 BD Pharmingen) was used at 0.005 mg/ml; a keratin antibody was used as the control. The detection system used was a commercially obtainable kit (Vectastain ABC AK-5002, Vector Labs).
   The results of these immunohistochemical investigations are shown in FIGS. 8A and B (TMEM45A) and C (SERPINB5/Maspin). The different staining confirmed that TMEM45A is expressed more strongly in cervical carcinomas than in normal cervix. This suggests that TMEM45A cannot only be used at the RNA level as a progression marker, but is also suitable at the protein level over antibodies.
   The same applies for SERPINB5, as here too it was possible to observe a different staining, which confirmed that SerpinB5 cannot only be used at the RNA level as a progression marker, but also at is suitable also at the protein level over antibodies as a prevalence marker.

### Discussion

It was possible to show by the present results that the genes tested and investigated are not only suitable surrogate markers for the presence of a persistent HPV infection, but also that a change in the gene expression of the markers determined is a prognostic marker for the development of cervical cancer and cancer precursors such as dysplasias.

Thus it was possible to show with the aid of the experiments shown above that, for example, the increase in the CDKN2A, SERPINB5 and TMEM45A RNA is a marker for an HPV infection and a marker for the prognosis of the development of cervical cancer.

It was furthermore possible to show that an increase in the amount of E6/E7 RNA, or an increase in the early viral transcription (E4 primer) is a prognostic marker for the progression of persistent HPV16-infected persons. The primer pairs suitable in this case are located in the 3′-region of the early viral transcription, so that additionally to the E6/E7 transcripts E1̂E4 transcripts can be detected.

It was furthermore possible to confirm that the identified genes are also suitable at the protein level as prevalence markers—for example by means of antibodies directed against these.

What is claimed is:

1. A method for predisposition prediction of a subject to the development of cervical cancer and/or cancer precursors in an infection with a human papilloma virus 16 (HPV16) and/or for the detection of a persistent HPV16 infection, the method comprising:
obtaining a sample from the subject;
detecting at least one of the diagnostic markers or fragments thereof listed in Table 1 in the sample obtained from the subject; and
determining that the subject will develop cervical cancer and/or cancer precursors in the infection with the HPV16 or detecting a persistent HPV16 infection in the subject,
wherein differential expression of at least one of the diagnostic markers in the sample obtained from the subject compared to a control determines that the subject will develop cervical cancer and/or cancer precursors in the infection with the HPV16 or detects the persistent HPV16 infection in the subject.

2. The method of claim 1, wherein the detecting step comprises analyzing the sample, by laboratory assay, for a change in the level of gene expression of the at least one diagnostic marker in the sample relative to the level of gene expression of a corresponding diagnostic marker in at least one control sample.

3. The method of claim 1, characterized in that the detecting step is carried out by means of the determination of the transcripts of the diagnostic markers and/or of the proteins encoded by the diagnostic markers.

4. The method of claim 1, wherein, for the detection of the diagnostic markers, at least one isolated oligonucleotide is used that is selected from the sequences listed in Table 3.

5. The method of claim 1, comprising the following steps:
(a) isolation of mRNA from a sample obtained from the subject;
(b) transcription of the mRNA isolated in step (a) to cDNA;
(c) bringing the cDNA sample obtained in step b) into contact with at least one oligonucleotide that is selected from the oligonucleotides listed in Table 3;
(d) carrying out a quantitative RT-PCR for the preparation of
(e) determination of the amplification products obtained in step (d).

6. The method of claim 5, characterized in that it comprises the additional step (f):
(f) comparison of the amplification products determined in step (e) with amplification products corresponding to these from corresponding comparison markers from an HPV16-negative sample and/or a corresponding HPV16-positive, but non-progressive sample, wherein a change, in particular a down- and/or up-regulation, of the gene expression of the diagnostic markers shows, in comparison to the comparison markers, the predisposition of a person to develop cervical cancer.

7. The method of claim 1, wherein reference markers are detected in parallel to the detection of the diagnostic markers.

8. The method of claim 1, wherein the sample is a tissue or body fluid sample of a human.

9. The method of claim 8, wherein the sample is a biopsy, a cervical sample, a cervical cell sample and/or a Papanicolaou smear of a human.

10. The method of claim 1, wherein the markers are selected from at least one of the group consisting of the markers listed in Table 2.

11. The method of claim 1, wherein one or more of the following markers are used: TMEM45A, SERPINB5 and CDKN2A.

12. The method of claim 1, wherein the method is carried out by the detection of a combination of two or more of the diagnostic markers listed in Table 1.

13. The method of claim 1, wherein for the detecting step an antibody is used, which is directed against at least one of the proteins that are expressed by the genes listed in Table 1.

14. The method of claim 1, wherein the subject has the persistent HPV16 infection, further comprising, treating the persistent HPV16 infection in the subject.

15. A method for determining if a subject is predisposed to developing cervical cancer following infection with a human papilloma virus 16 (HPV 16), comprising:
quantitating the amount of mRNA encoding TMEM45A in a biological sample from the subject in vitro;
comparing the amount of mRNA in the biological sample to a control, and
determining that the subject is predisposed to developing cervical cancer following infection with the HPV16, wherein an increase in the amount of mRNA encoding TMEM45A in the sample as compared to the control indicates that the subject is predisposed to developing cervical cancer following infection with the HPV16.

16. The method of claim 15, wherein quantitating the amount of mRNA encoding TEM45A comprises the use of real time polymerase chain reaction (RT-PCR).

17. The method of claim 16, wherein the method comprises
contacting the biological sample with a primer comprising the nucleic acid sequence set forth as SEQ ID NO: 20 or its complement, and a primer comprising the nucleic acid sequence set forth as SEQ ID NO: 21 or its complement.

18. The method of claim 15, wherein the mRNA encoding TMEM45A comprises a nucleic acid sequence at least 95% identical to SEQ ID NO: 19.

19. The method of claim 15, further comprising
quantitating the amount of an mRNA encoding human papilloma virus E4, an mRNA encoding human papilloma virus E6 and/or an mRNA encoding human papilloma virus E7.

20. The method of claim 15, wherein the method comprises the use of a microarray.

21. The method of claim 15, wherein the biological sample is a cervical sample.

22. The method of claim 15, further comprising treating the HPV16 infection in the subject.

23. An isolated or purified oligonucleotide for detecting at least one diagnostic marker represented in Table 2, wherein the oligonucleotide is selected from at least one of the following:
(a) an oligonucleotide listed in Table 3 or its complementary strand;
(b) oligonucleotides that hybridize under stringent conditions to the oligonucleotides defined in (a), or fragments thereof; and
(c) oligonucleotides that have a sequence homology of at least 80%, preferably of at least 85%, 90%, 95% or 98% to the oligonucleotides defined in (a) and with which the diagnostic markers can likewise be detected.


SEQUENCE LISTING (51 sequences)

SEQ ID NO: 1 - DNA, 547 bp, Homo sapiens
cttcgttcgcagagcttttcagattgtggaatgttggataaggaattatagacctctagtagctgaaatgcaagaccccaagaggaagttcagatcttaatataaattcactttcatttttgatagctgtcccatctggtcatgtggttggcactagactggtggcaggggcttctagctgactcgcacagggattctcacaatagccgatatcagaatttgtgttgaaggaacttgtctcttcatctaatatgatagcgggaaaaggagaggaaactactgcctttagaaaatataagtaaagtgattaaagtgctcacgttaccttgacacatagtttttcagtctatgggtttagttactttagatggcaagcatgtaacttatattaatagtaatttgtaaagttgggtggataagctatccctgttgccggttcatggattacttctctataaaaaatatatatttaccaaaaaattttgtgacattccttctcccatctcttccttgacatgcattgtaaataggttcttcttgttctgag

SEQ ID NO: 2 - DNA, 20 bp, Artificial Sequence
  forward Primer SERPINB5
gttgccggttcatggattac

SEQ ID NO: 3 - DNA, 20 bp, Artificial Sequence
  reverse Primer SERPINB5
gcatgtcaaggaagagatgg

SEQ ID NO: 4 - DNA, 370 bp, Homo sapiens
gaggcagttgttaaattgagtgaccaatttaagcaatcagatatttgaaaactgcaccctttagttttgaaactgtgaattagaaacacttttcctgctgtattactacctgctttaacatccaaatatacagtgattttaaatgataacatactgtggttattagattaacagcttgattttgaatgttcagatgataatgcagaagacatcacttctagtaaggattttgactagtgcattgatgttgaagttggtgccatttcaaaatgtggcaggtgataatcttttaccataatttgcataaaactgtaatagaagtttattttgagatgttagtatattatgtactatgcatttctgtggtata

SEQ ID NO: 5 - DNA, 23 bp, Artificial Sequence
  forward Primer CAND1
gcttgattttgaatgttcagatg

SEQ ID NO: 6 - DNA, 22 bp, Artificial Sequence
  reverse Primer CAND1
gattatcacctgccacattttg

SEQ ID NO: 7 - DNA, 360 bp, Homo sapiens
  misc_feature (201)..(206): n is a, c, g, or t
aagcatacgtgctatgactctctccagtttgaatatgcaattgttttcacaggcaggatgtctgttttctgcctgtatttcccagtgatttactctagggtaaggtagtacacatttggttcagaaattaatttttatttctcctatatcttgttttatcaagattttgttgtggcatttcaatgtaaattataacaccannnnnntcatttgagtatacataattcaaaagaactacttgatgcagtatagtcttaagggttctgcatacattttagaaacatcttagccgtaagttaggtcctgtgttaaactgtttagtgctctgtttttaagaaaacaaatgttgaacctcacact

SEQ ID NO: 8 - DNA, 22 bp, Artificial Sequence
  forward Primer CPSF2
gccgtaagttaggtcctgtgtt

SEQ ID NO: 9 - DNA, 20 bp, Artificial Sequence
  reverse Primer CPSF2
tgcttgggtgaccttaggag

SEQ ID NO: 10 - DNA, 531 bp, Homo sapiens
ctctcctgtatgcgtttcttgatgggacatttagcaaatacctctgccgctgtttccatctgcgtagtaacaccccacttcaacccagggggcagtctgcacaaggcacatcgagggaagaacctgaccattccaccgaagtgtaaactagcatccaccaaatgcaagaagaataaacatggattttcatctttctgcattatttcatgtaaattttctacacatttgtatacaaaatcggatacaggaagaaaagggagaggtgagctaacatttgctaagcactgaatttgtctcaggcaccgtgcaaggctctttacaaacgtgagctccttcgcctcctaccacttgtccatagtgtggataggactagtctcatttctctgagaagaaaactaaggcgcggaaatttgtctaagatcacataactaggaagtggcagaactgattctccagccctggtagcatttgctcagagcctacgcttggtccagaacatcaaactccaaaccctggggacaaacgacatga

SEQ ID NO: 11 - DNA, 20 bp, Artificial Sequence
  forward Primer CCRL2
ggcgcggaaatttgtctaag

SEQ ID NO: 12 - DNA, 20 bp, Artificial Sequence
  reverse Primer CCRL2
ccagggtttggagtttgatg

SEQ ID NO: 13 - DNA, 490 bp, Homo sapiens
ggataccccattgtgaagccagggcccaactgtggatttggaaaaactggcattattgattatggaatccgtctcaataggagtgaaagatgggatgcctattgctacaacccacacgcaaaggagtgtggtggcgtctttacagatccaaagcaaatttttaaatctccaggcttcccaaatgagtacgaagataaccaaatctgctactggcacattagactcaagtatggtcagcgtattcacctgagttttttagattttgaccttgaagatgacccaggttgcttggctgattatgttgaaatatatgacagttacgatgatgtccatggctttgtgggaagatactgtggagatgagcttccagatgacatcatcagtacaggaaatgtcatgaccttgaagtttctaagtgatgcttcagtgacagctggaggtttccaaatcaaatatgttgcaatggatcctgtatccaaatccagtcaag

SEQ ID NO: 14 - DNA, 20 bp, Artificial Sequence
  forward Primer TNFAIP6
ttgaagatgacccaggttgc

SEQ ID NO: 15 - DNA, 21 bp, Artificial Sequence
  reverse Primer TNFAIP6
catctggaagctcatctccac

SEQ ID NO: 16 - DNA, 562 bp, Homo sapiens
ctggatcaccgacagcacagacagaatccctgctaccagagaccaagacacattccaccccagtggggggtcccataccactcatgaatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttgtaaacataaccattacagggagctgggacacttaacagatgcaatgtgctactgattgtttcattgcga

SEQ ID NO: 17 - DNA, 20 bp, Artificial Sequence
  forward Primer CD44
aaggtggagcaaacacaacc

SEQ ID NO: 18 - DNA, 20 bp, Artificial Sequence
  reverse Primer CD44
gctttttcttctgcccacac

SEQ ID NO: 19 - DNA, 563 bp, Homo sapiens
tgtttctcaccatatgcttttgttggcattatgcagtaaccattgtcatcgttggaatgaattatgctttcattacctggttggttaaatctagacttaagaggctctgctcctcagaagttggacttctgaaaaatgctgaacgagaacaagaatcagaagaagaaatgtgactttgatgagcttccagtttttctagataaaccttttcttttttacattgttcttggttttgtttctcgatcttttgtttggagaacagctggctaaggatgactctaagtgtactgtttgcatttccaatttggttaaagtatttgaatttaaatattttctttttagctttgaaaatattttgggtgatactttcattttgcacatcatgcacatcatggtattcaggggctagagtgatttttttccagattatctaaagttggatgcccacactatgaaagaaatatttgttttatttgccttatagatatgctcaaggttactgggcttgctactatttgtaactccttgaccatggaattatacttgtttatcttgttgctgca

SEQ ID NO: 20 - DNA, 20 bp, Artificial Sequence
  forward Primer TMEM45A
agttggatgcccacactatg

SEQ ID NO: 21 - DNA, 22 bp, Artificial Sequence
  reverse Primer TMEM45A
agtagcaagcccagtaaccttg

SEQ ID NO: 22 - DNA, 276 bp, Homo sapiens
  misc_feature (125)..(125): n is a, c, g, or t
gctcactaggggacatgcacatggaagctcacctctaagaaagggctgggcagatggatttattttttcccacctgtgaatatgtaaaacaaaaccattatctttgagggagtttttaataccantgacagaacagagatttgtgtgctcatcttaagagccagagccatataagcatcttgggaaagcaagtttgaaccagctgctggtgtaatgtacagttatatttgtctataaatggagctgtttatggcaatttaataccattctctttgt

SEQ ID NO: 23 - DNA, 20 bp, Artificial Sequence
  forward Primer 43A
acatgcacatggaagctcac

SEQ ID NO: 24 - DNA, 20 bp, Artificial Sequence
  reverse Primer WDR43
aacttgctttcccaagatgc

SEQ ID NO: 25 - DNA, 561 bp, Homo sapiens
  misc_feature (135)..(135): n is a, c, g, or t
agcgccccagtgaacactacaacatacgtagctgacacagaatcagagcaagcagatacatgggatttgagtgaaaggccaaaagaaatcaaagtctccaaaatggaacaaaaattcagaatgctttcacaagangcacccactgtaaaggagtcctgcaaaacaagctctaataataatagtatggtatcaaatactttggctaagatgagaatcccaaactatcagctttcaccaactaaattgccaagtataaataaaagtaaagatagggcttctcagcagcagcagaccaactccatcagaaactactttcagccgtctaccaaaaaaagggaaagggatgaagaaaatcaagaaatgtcttcatgcaaatcagcaagaatagaaacgtcttgttctcttttagaacaaacacaacctgctacaccctcattgtggaaaaataaggagcagcatctatctgagaatgagcctgtggacacaaactcagacaataacttatttacagatacagatttaaaatctattgtgaaaaattctgccagtaaatctcatgct

SEQ ID NO: 26 - DNA, 20 bp, Artificial Sequence
  forward Primer NBN
atagggcttctcagcagcag

SEQ ID NO: 27 - DNA, 20 bp, Artificial Sequence
  reverse Primer NBN
tccacaatgagggtgtagca

SEQ ID NO: 28 - DNA, 553 bp, Homo sapiens
gaagctttggtattgtacaaggtcagtagtaagatgctcactagtctcagggcttgtgtaatattctgggaggtcatttaaatgccagaaatggtcaagcaattatacacagtatttatgactctgttaagcataccgtttgtctgtcacattagtagattctgagattaaaaaaaatttttaaagagtgatcatttaaataatttctaaaagggtcttttcaagctctaacaaagtcactaacaaatgcattattttctacagaattagatgttagtagtacagtactgcatattcagggaaaaagtgtgaggaattgatttcaaaatagttcgttcttgtgtttgacctaagaatgattgtcgcatgaagtgtttgtttttacagtttagcatatataaacaaacatgataggattccttaagatgttaccacccagggggccacaagccagcctgctgtctcaggaagctgtagaaggagtgtttgtcaatttcttgtcactggtttgctgacttactgaggattaattgttgccttacaatgttactga

SEQ ID NO: 29 - DNA, 20 bp, Artificial Sequence
  forward Primer ERMP1
aatgccagaaatggtcaagc

SEQ ID NO: 30 - DNA, 21 bp, Artificial Sequence
  reverse Primer
ttcctcacactttttccctga

SEQ ID NO: 31 - DNA, 520 bp, Homo sapiens
  misc_feature (50)..(50): n is a, c, g, or t
gtatggtgcctatctacactcacatgatattcttattcacgttttttttnaaccataagtggcaaatattttaaaatatttgaaaaacactccagaatctagtacgctttatttttagactgaacctaaagtaggttgttcttttaacaaagggtttaattcgggtggggaatataacatatcaaaatacatgaacaaatggaaagttacttctagaaaagcaaagaaattgggtatcatttttgtttcttgggaagctaattttgttgaatgtttagaattgagcaaagatgtaaatttttgaagggcagtttagaaaaattaactttgtgaatgaacttaagatgtctgtactctatatgtgatgctgtgcagtttgtttttatatggaaagatgtcaactatagccataaccaataaaataaaaactgatgaggcatgcagctttcagcacatcttttatacatgaagaaattaattttgtgttgctatggtgttgaaatatccaagatgttctgta

SEQ ID NO: 32 - DNA, 20 bp, Artificial Sequence
  forward Primer LRP11
ttcgggtggggaatataaca

SEQ ID NO: 33 - DNA, 20 bp, Artificial Sequence
  reverse Primer LRP11
acaaactgcacagcatcaca

SEQ ID NO: 34 - DNA, 110 bp, Human Papillomavirus
caccgaagaaacacagacgactatccagcgaccaagatcagagccagacaccggaaacccctgccacaccactaagttgttgcacagagactcagtggacagtgctccaa

SEQ ID NO: 35 - DNA, 20 bp, Artificial Sequence
  forward Primer HPV16E4
caccgaagaaacacagacga

SEQ ID NO: 36 - DNA, 20 bp, Artificial Sequence
  reverse Primer HPV16E4
ttggagcactgtccactgag

SEQ ID NO: 37 - DNA, 72 bp, Human Papillomavirus
gacaagcagaaccggacagagcccattacaatattgtaaccttttgttgcaagtgtgactctacgcttcggt

SEQ ID NO: 38 - DNA, 20 bp, Artificial Sequence
  forward Primer HPV16E7
gacaagcagaaccggacaga

SEQ ID NO: 39 - DNA, 20 bp, Artificial Sequence
  reverse Primer HPV16E7
accgaagcgtagagtcacac

SEQ ID NO: 40 - DNA, 443 bp, Homo sapiens
gaggaggcaggcgagtgagcgagtccgaggggtggccggggcaggtggtggcgccgcgaagatggtcgccaagcaaaggatccgtatggccaacgagaagcacagcaagaacatcacccagcgcggcaacgtcgccaagacctcgagaaatgcccccgaagagaaggcgtctgtaggaccctggttattggctctcttcatttttgttgtctgtggttctgcaattttccagattattcaaagtatcaggatgggcatgtgaagtgactgaccttaagatgtttccattctcctgtgaatttttaacttgaactcattcctgatgtttgataccctggttgaaaacaattcagtaaagcatcctgcctcagaatgactttcctatcatgcttcatgtgtcattccaaggtttcttcatgagtcattccaagttttctagtcca

SEQ ID NO: 41 - DNA, 22 bp, Artificial Sequence
  forward Primer SERP1
tctgtaggaccctggttattgg

SEQ ID NO: 42 - DNA, 20 bp, Artificial Sequence
  reverse Primer SERP1
tcacatgcccatcctgatac

SEQ ID NO: 43 - DNA, 531 bp, Homo sapiens
  misc_feature (71)..(74): n is a, c, g, or t
gagggatgagacagacattcacctgtatatttcttttaatgggcacaaaatgggcccttgcctctaaatannnntnnnnngnnttnaagaagtaatcagtatgcaaagcaatcttttatacaataattgaagtgttnccntttttcataattactctacttcccagtaaccctaaggaagttgctaacttaaaaaanctgcatcccacgttctgttaatttagtaaataaacaagtcaaagacttgtggaaaataggaagtgaacccatattttaaattctcataagtagcattcatgtaataaacaggtttttagtttgttcttcagattgatagggagttttaaagaaattttagtagttactaaaattatgttactgtatttttcagaaatcnaactgcttatgaaaagtactaatagaacttgttaacctttctaaccttcacgattaactgtgaaatgtacgtcatttgtgcaagaccgtttgtccacttcattttgtataatcacagttgtgttcctgacactca

SEQ ID NO: 44 - DNA, 22 bp, Artificial Sequence
  forward Primer ASAH1
cccagtaaccctaaggaagttg

SEQ ID NO: 45 - DNA, 22 bp, Artificial Sequence
  reverse Primer ASAH1
gggttcacttcctattttccac

SEQ ID NO: 46 - DNA, 540 bp, Homo sapiens
ttaattcaactttcgtctgcctgttttgtggactggctggctctgttagaactctgtccaaaaagtgcatggaatataacttgtaaagcttcccacaattgacaatatatatgcatgtgtttaaaccaaatccagaaagcttaaacaatagagctgcataatagtatttattaaagaatcaccaactgtaaacatgagaataacttaaggttctagtttagttttttgtaattgcaaattatatttttgctgctgatatattagaataattttaaaatgtcatcttgaaatagaaatatgtattttaagcactcacgcaaaggtaaatgaacacgttttaaatgtgtgtgttgctaattttttccataagaattgtaaacattgaactgaacaaattacctataatggatttggttaatgacttatgagcaagctggtttggccagacagtatacccaaacttttatataatatacagaaggctatcacacttgtgaaattctcttgtctaatctgaatttgcattccatggtgttaaca

SEQ ID NO: 47 - DNA, 21 bp, Artificial Sequence
  forward Primer HIGD1A
cttatgagcaagctggtttgg

SEQ ID NO: 48 - DNA, 21 bp, Artificial Sequence
  reverse Primer HIGD1A
caccatggaatgcaaattcag

SEQ ID NO: 49 - DNA, 198 bp, Homo sapiens
ctgtgggggtatttgaatgggaagcttttgcccggggaaccaaagctctcatggatgaggtggtgaaagccacttctaggggctgcatcaccatcataggtggtggagacactgccacttgctgtgccaaatggaacacggaggataaagtcagccatgtgagcactgggggtggtgccagtttggagctcctggaag

SEQ ID NO: 50 - DNA, 20 bp, Artificial Sequence
  forward Primer PGK1
ctgtgggggtatttgaatgg

SEQ ID NO: 51 - DNA, 20 bp, Artificial Sequence
  reverse Primer PGK1
cttccaggagctccaaactg